(19) United States
(12) Patent Application Publication
(10) Pub. No.: US 2021/0327089 A1
(43) Pub. Date: Oct. 21, 2021

(54) Method for Measuring Positions

(71) Applicant: Ying Ji, Zhejiang (CN)

(72) Inventor: Ying Ji, Zhejiang (CN)

(21) Appl. No.: 17/272,563

(22) PCT Filed: Aug. 27, 2018
(86) PCT No.: PCT/CN2018/102538

§ 371 Date: Mar. 1, 2021

(51) Int. Cl. G06T 7/73; A61B 34/20

(52) U.S. Cl. CPC G06T 7/74; A61B 34/20; G06T 2207/30204; A61B 2090/3954; A61B 2034/2065; A61B 2034/2051; A61B 2034/2072

(57) ABSTRACT

A method of measuring a target's position is provided. The method comprises: providing a marker for the target and a tracking assembly. The marker has a convex measuring surface, configured to be part or whole of a sphere, such that the center of the convex measuring surface substantially corresponds to the position of the target. The tracking assembly comprises a measuring piece, which has a tracking tool fixedly attached onto. One type of the measuring piece has a concave measuring surface substantially fit with the convex measuring surface of the marker; Another type of the measuring piece comprises a vision measuring system configured to be able to measure position of a center of the marker with respect to a designated coordinate system of the vision measuring system. The method to obtain the calibration relationship between the designated coordinate system of the vision measuring system and the tracking tool is described also. The disclosed method is more convenient and is able to improve the accuracy for measuring a target position.

### BACKGROUND

When using a surgical navigation system to assist an operation, markers are typically placed around a patient (for example, on the skin of a patient). After CT/MRI scanning, these markers are commonly shown in the resulting images. In order to correlate the markers in the CT/MR images with markers around the patient in a physical world, a registering is typically performed with a register pen. The so-called register pen is usually used along with a tracking assembly to measure the positions of these markers in the physical world. Each of these markers is usually considered as a dot. By correlating the dot's position in the physical world with that in the imaging world, the transformation is obtained so as to convert any position in the physical world into that in the imaging world. Then during a surgical procedure aided with a navigation system, a medical instrument with its position tracked by a tracking assembly can be displayed in the image with the scanned patient images together.

### SUMMARY

The inventor of the present disclosure has recognized that there are inaccuracies associated with the current approach for determining a position of a marker during a registering.

The present disclosure relates generally to the field of target tracking, and more specifically to a method for measuring positions.

In some embodiments, a method of measuring at least one target's position comprising:
a) providing a marker for each target and a tracking assembly, wherein: each marker has a convex measuring surface, configured to be part or whole of a sphere, such that the center of the convex measuring surface substantially corresponds to the position of the target to be measured; and the tracking assembly comprises a measuring piece; and the tracking assembly further comprises a tracking tool, fixedly attached onto the measuring piece; and the measuring piece is configured to be able to obtain the center position of the convex measuring surface of the marker/markers with respect to the tracking tool; and the tracking assembly is configured to be able to obtain the tracking tool's position and direction data with respect to a reference coordinate system of the tracking assembly;
b) obtaining and recording the center position data of the convex measuring surface of each of the at least one marker with respect to the tracking tool and the tracking tool's position and orientation data with respect to the reference coordinate system of the tracking assembly;
c) calculating, based on the recorded center position data of the convex measuring surface of each of the at least one marker with respect to the tracking tool, and the recorded tracking tool's position and orientation data with respect to the reference coordinate system of the tracking assembly, to thereby obtain each target's the position with respect to the reference coordinate system of the tracking assembly.

In some embodiments, the measuring piece has a concave measuring surface substantially fit with the convex measuring surface of each of the at least one marker; and the measuring piece is configured to be able to obtain the center position of the concave surface with respect to the tracking tool.

In some embodiments, in step b), the obtaining and recording the center position data of the convex measuring surface of each of the at least one marker with respect to the tracking tool and the tracking tool's position and orientation data with respect to the reference coordinate system of the tracking assembly is by contacting the concave surface of the measuring piece with the convex measuring surface of each of the at least one marker.

In some embodiments, the measuring piece comprises differently a vision measuring system configured to be able to measure position of a center of each of the at least one marker with respect to a designated coordinate system of the vision measuring system; and the calibration relationship between the designated coordinate system of the vision measuring system and the tracking tool is known;

Therefore, the obtaining and recording the center position data of the convex measuring surface of each of the at least one marker with respect to the tracking tool is based on the measured position of a center of each of the at least one marker with respect to the designated coordinate system of the vision measuring system and the calibration relationship between the designated coordinate system of the vision measuring system and the tracking tool.

Mathematically, the position of a center of each of the at least one marker with respect to the designated coordinate system of the vision measuring system is expressed as (x_b, y_b, z_b), satisfying a relationship:

$$( \begin{matrix}x \_ s \\ y \_ s \\ z \_ s\end{matrix} ) = ( \begin{matrix}Δ x \\ Δ y \\ Δ z\end{matrix} ) + ( \begin{matrix}{r}_{00} & {r}_{01} & {r}_{02} \\ {r}_{10} & {r}_{11} & {r}_{12} \\ {r}_{20} & {r}_{21} & {r}_{22}\end{matrix} ) ( \begin{matrix}x \_ b \\ y \_ b \\ z \_ b\end{matrix} ) \qquad ( 1 )$$

wherein: the (Δx, Δy, Δz)^{T} represents an offset between a zero point of the designated coordinate system of the vision measuring system and the position of the tracking tool;

the 3×3 matrix:

$$( \begin{matrix}{r}_{00} & {r}_{01} & {r}_{02} \\ {r}_{10} & {r}_{11} & {r}_{12} \\ {r}_{20} & {r}_{21} & {r}_{22}\end{matrix} )$$

represents a rotational relationship between the designated coordinate system of the vision measuring system and the tracking tool; and the (x_s, y_s, z_s) represents the center position of each of the at least one marker with respect to the tracking tool.
Similarly, the center position of each of the at least one marker with respect to the tracking tool expressed as (x_s, y_s, z_s) is further satisfied with a relationship:

$$( \begin{matrix}x \_ t \\ y \_ t \\ z \_ t\end{matrix} ) = ( \begin{matrix}{x}^{′} \\ {y}^{′} \\ {z}^{′}\end{matrix} ) + ( \begin{matrix}{m}_{00} & {m}_{01} & {m}_{02} \\ {m}_{10} & {m}_{11} & {m}_{12} \\ {m}_{20} & {m}_{21} & {m}_{22}\end{matrix} ) ( \begin{matrix}x \_ s \\ y \_ s \\ z \_ s\end{matrix} ) \qquad ( 2 )$$

wherein: the (x′, y′, z′)T represents a position of the tracking tool with regard to the tracking assembly's coordinate system; and the 3×3 matrix:

$$( \begin{matrix}{m}_{00} & {m}_{01} & {m}_{02} \\ {m}_{10} & {m}_{11} & {m}_{12} \\ {m}_{20} & {m}_{21} & {m}_{22}\end{matrix} )$$

represents a rotational relationship between tracking tool and the tracking assembly's coordinate system; and the (x_t, y_t, z_t) represents a center position of each of the at least one marker with respect to the tracking assembly's coordinate system.

Further mover, the calculating, based on the recorded center position data of the convex measuring surface of each of the at least one marker with respect to the tracking tool, and the recorded tracking tool's position and orientation data with respect to the reference coordinate system of the tracking assembly, to thereby obtain each target's the position with respect to the reference coordinate system of the tracking assembly comprises:

substituting (x_s, y_s, z_s)T in formula (2) with (x_s, y_s, z_s)T in formula (1) to obtain a formula

$$( \begin{matrix}{x}_{t} \\ {y}_{t} \\ {z}_{t}\end{matrix} ) = ( \begin{matrix}{x}^{′} \\ {y}^{′} \\ {z}^{′}\end{matrix} ) + ( \begin{matrix}{m}_{00} & {m}_{01} & {m}_{02} \\ {m}_{10} & {m}_{11} & {m}_{12} \\ {m}_{20} & {m}_{21} & {m}_{22}\end{matrix} ) ( \begin{matrix}Δ x \\ Δ y \\ Δ z\end{matrix} ) + ( \begin{matrix}{m}_{00} & {m}_{01} & {m}_{02} \\ {m}_{10} & {m}_{11} & {m}_{12} \\ {m}_{20} & {m}_{21} & {m}_{22}\end{matrix} ) ( \begin{matrix}{r}_{00} & {r}_{01} & {r}_{02} \\ {r}_{10} & {r}_{11} & {r}_{12} \\ {r}_{20} & {r}_{21} & {r}_{22}\end{matrix} ) ( \begin{matrix}x \_ b \\ y \_ b \\ z \_ b\end{matrix} ) \qquad ( 3 )$$

to thereby calculate the position (x_t, y_t, z_t) of core center of each of the at least one maker in the space with respect to the reference coordinate system of the tracking assembly.

It is noted the calibration relationship between the designated coordinate system of the vision measuring system and the tracking tool is known by a method. The method to determine the calibration relationship comprising:
a) providing at least one marker and a tracking assembly, wherein: each of the at least one marker is provided with a convex measuring surface configured to be part or whole of a sphere; and the tracking assembly comprises a vision measuring system configured to be able to measure center position of each of the at least one marker with respect to the designated coordinate system of the vision measuring system; and the tracking assembly further comprises a tracking tool, fixedly attached onto the vision measuring system; and the tracking assembly is configured to be able to obtain the tracking tool's position and direction data with respect to a reference coordinate system of the tracking assembly;
b) arranging a number of N marker/markers such that each relative position between the center position of each marker and the same origin point of the reference coordinate system of the tracking assembly is fixed, wherein N≥1;
c) placing the vision measuring system at at least the number of p different positions relative to the reference origin point of the tracking assembly, and recording different relative center position data of the number of N marker/markers with respect to the designated coordinate system of the vision measuring system via the vision measuring system and position and orientation data of the tracking tool corresponding to each of the at least p different positions via the tracking assembly, wherein p=5 if N=1, p=3 if N=2 or N=3, and p=2 if N≥4; and
d) solving, based on the at least p groups of relative center position data of N marker/markers, nonhomogeneous linear equations to thereby obtain calibration relationship between the designated coordinate system of the vision measuring system and the tracking tool, wherein the nonhomogeneous linear equations are derived from the relationship between a center position of spherical marker with respect to the designated coordinate system of the vision measuring system and that position with respect to the coordinate system of the tracking assembly.

In step c), the placing the vision measuring system at at least the number of p different positions relative to the reference origin point of the tracking assembly, and recording different relative center position data of the number of N marker/markers with respect to the designated coordinate system of the vision measuring system via the vision measuring system and position and orientation data of the tracking tool corresponding to each of the at least p different positions via the tracking assembly comprises:

obtaining at least p×3×N equations in at least p×N equation groups:

$$( \begin{matrix}{x}_{\_ t}^{1} \\ {y}_{\_ t}^{1} \\ {z}_{\_ t}^{1}\end{matrix} ) = ( \begin{matrix}{x}_{1}^{′} \\ {y}_{1}^{′} \\ {z}_{1}^{′}\end{matrix} ) ( \begin{matrix}{m}_{00}^{1} & {m}_{01}^{1} & {m}_{02}^{1} \\ {m}_{10}^{1} & {m}_{11}^{1} & {m}_{12}^{1} \\ {m}_{20}^{1} & {m}_{21}^{1} & {m}_{22}^{1}\end{matrix} ) ( \begin{matrix}Δ x \\ Δ y \\ Δ z\end{matrix} ) + ( \begin{matrix}{m}_{00}^{1} & {m}_{01}^{1} & {m}_{02}^{1} \\ {m}_{10}^{1} & {m}_{11}^{1} & {m}_{12}^{1} \\ {m}_{20}^{1} & {m}_{21}^{1} & {m}_{22}^{1}\end{matrix} ) ( \begin{matrix}{r}_{00} & {r}_{01} & {r}_{02} \\ {r}_{10} & {r}_{11} & {r}_{12} \\ {r}_{20} & {r}_{21} & {r}_{22}\end{matrix} ) ( \begin{matrix}{x}_{\_ b 1}^{1} \\ {y}_{\_ b 1}^{1} \\ {z}_{\_ b 1}^{1}\end{matrix} ) \\ ( \begin{matrix}{x}_{\_ t}^{2} \\ {y}_{\_ t}^{2} \\ {z}_{\_ t}^{2}\end{matrix} ) = ( \begin{matrix}{x}_{1}^{′} \\ {y}_{1}^{′} \\ {z}_{1}^{′}\end{matrix} ) ( \begin{matrix}{m}_{00}^{1} & {m}_{01}^{1} & {m}_{02}^{1} \\ {m}_{10}^{1} & {m}_{11}^{1} & {m}_{12}^{1} \\ {m}_{20}^{1} & {m}_{21}^{1} & {m}_{22}^{1}\end{matrix} ) ( \begin{matrix}Δ x \\ Δ y \\ Δ z\end{matrix} ) + ( \begin{matrix}{m}_{00}^{1} & {m}_{01}^{1} & {m}_{02}^{1} \\ {m}_{10}^{1} & {m}_{11}^{1} & {m}_{12}^{1} \\ {m}_{20}^{1} & {m}_{21}^{1} & {m}_{22}^{1}\end{matrix} ) ( \begin{matrix}{r}_{00} & {r}_{01} & {r}_{02} \\ {r}_{10} & {r}_{11} & {r}_{12} \\ {r}_{20} & {r}_{21} & {r}_{22}\end{matrix} ) ( \begin{matrix}{x}_{\_ b 2}^{1} \\ {y}_{\_ b 2}^{1} \\ {z}_{\_ b 2}^{1}\end{matrix} ) \\ ………… \\ ( \begin{matrix}{x}_{\_ t}^{N} \\ {y}_{\_ t}^{N} \\ {z}_{\_ t}^{N}\end{matrix} ) = ( \begin{matrix}{x}_{1}^{′} \\ {y}_{1}^{′} \\ {z}_{1}^{′}\end{matrix} ) ( \begin{matrix}{m}_{00}^{1} & {m}_{01}^{1} & {m}_{02}^{1} \\ {m}_{10}^{1} & {m}_{11}^{1} & {m}_{12}^{1} \\ {m}_{20}^{1} & {m}_{21}^{1} & {m}_{22}^{1}\end{matrix} ) ( \begin{matrix}Δ x \\ Δ y \\ Δ z\end{matrix} ) + ( \begin{matrix}{m}_{00}^{1} & {m}_{01}^{1} & {m}_{02}^{1} \\ {m}_{10}^{1} & {m}_{11}^{1} & {m}_{12}^{1} \\ {m}_{20}^{1} & {m}_{21}^{1} & {m}_{22}^{1}\end{matrix} ) ( \begin{matrix}{r}_{00} & {r}_{01} & {r}_{02} \\ {r}_{10} & {r}_{11} & {r}_{12} \\ {r}_{20} & {r}_{21} & {r}_{22}\end{matrix} ) ( \begin{matrix}{x}_{\_ bN}^{1} \\ {y}_{\_ bN}^{1} \\ {z}_{\_ bN}^{1}\end{matrix} ) \\ ( \begin{matrix}{x}_{\_ t}^{1} \\ {y}_{\_ t}^{1} \\ {z}_{\_ t}^{1}\end{matrix} ) = ( \begin{matrix}{x}_{2}^{′} \\ {y}_{2}^{′} \\ {z}_{2}^{′}\end{matrix} ) ( \begin{matrix}{m}_{00}^{2} & {m}_{01}^{2} & {m}_{02}^{2} \\ {m}_{10}^{2} & {m}_{11}^{2} & {m}_{12}^{2} \\ {m}_{20}^{2} & {m}_{21}^{2} & {m}_{22}^{2}\end{matrix} ) ( \begin{matrix}Δ x \\ Δ y \\ Δ z\end{matrix} ) + ( \begin{matrix}{m}_{00}^{2} & {m}_{01}^{2} & {m}_{02}^{2} \\ {m}_{10}^{2} & {m}_{11}^{2} & {m}_{12}^{2} \\ {m}_{20}^{2} & {m}_{21}^{2} & {m}_{22}^{2}\end{matrix} ) ( \begin{matrix}{r}_{00} & {r}_{01} & {r}_{02} \\ {r}_{10} & {r}_{11} & {r}_{12} \\ {r}_{20} & {r}_{21} & {r}_{22}\end{matrix} ) ( \begin{matrix}{x}_{\_ b 1}^{2} \\ {y}_{\_ b 1}^{2} \\ {z}_{\_ b 1}^{2}\end{matrix} ) \\ ( \begin{matrix}{x}_{\_ t}^{2} \\ {y}_{\_ t}^{2} \\ {z}_{\_ t}^{2}\end{matrix} ) = ( \begin{matrix}{x}_{2}^{′} \\ {y}_{2}^{′} \\ {z}_{2}^{′}\end{matrix} ) ( \begin{matrix}{m}_{00}^{2} & {m}_{01}^{2} & {m}_{02}^{2} \\ {m}_{10}^{2} & {m}_{11}^{2} & {m}_{12}^{2} \\ {m}_{20}^{2} & {m}_{21}^{2} & {m}_{22}^{2}\end{matrix} ) ( \begin{matrix}Δ x \\ Δ y \\ Δ z\end{matrix} ) + ( \begin{matrix}{m}_{00}^{2} & {m}_{01}^{2} & {m}_{02}^{2} \\ {m}_{10}^{2} & {m}_{11}^{2} & {m}_{12}^{2} \\ {m}_{20}^{2} & {m}_{21}^{2} & {m}_{22}^{2}\end{matrix} ) ( \begin{matrix}{r}_{00} & {r}_{01} & {r}_{02} \\ {r}_{10} & {r}_{11} & {r}_{12} \\ {r}_{20} & {r}_{21} & {r}_{22}\end{matrix} ) ( \begin{matrix}{x}_{\_ b 2}^{2} \\ {y}_{\_ b 2}^{2} \\ {z}_{\_ b 2}^{2}\end{matrix} ) \\ ………… \\ ( \begin{matrix}{x}_{\_ t}^{N} \\ {y}_{\_ t}^{N} \\ {z}_{\_ t}^{N}\end{matrix} ) = ( \begin{matrix}{x}_{2}^{′} \\ {y}_{2}^{′} \\ {z}_{2}^{′}\end{matrix} ) ( \begin{matrix}{m}_{00}^{2} & {m}_{01}^{2} & {m}_{02}^{2} \\ {m}_{10}^{2} & {m}_{11}^{2} & {m}_{12}^{2} \\ {m}_{20}^{2} & {m}_{21}^{2} & {m}_{22}^{2}\end{matrix} ) ( \begin{matrix}Δ x \\ Δ y \\ Δ z\end{matrix} ) + ( \begin{matrix}{m}_{00}^{2} & {m}_{01}^{2} & {m}_{02}^{2} \\ {m}_{10}^{2} & {m}_{11}^{2} & {m}_{12}^{2} \\ {m}_{20}^{2} & {m}_{21}^{2} & {m}_{22}^{2}\end{matrix} ) ( \begin{matrix}{r}_{00} & {r}_{01} & {r}_{02} \\ {r}_{10} & {r}_{11} & {r}_{12} \\ {r}_{20} & {r}_{21} & {r}_{22}\end{matrix} ) ( \begin{matrix}{x}_{\_ bN}^{2} \\ {y}_{\_ bN}^{2} \\ {z}_{\_ bN}^{2}\end{matrix} ) \\ ( \begin{matrix}{x}_{\_ t}^{1} \\ {y}_{\_ t}^{1} \\ {z}_{\_ t}^{1}\end{matrix} ) = ( \begin{matrix}{x}_{p}^{′} \\ {y}_{p}^{′} \\ {z}_{p}^{′}\end{matrix} ) ( \begin{matrix}{m}_{00}^{p} & {m}_{01}^{p} & {m}_{02}^{p} \\ {m}_{10}^{p} & {m}_{11}^{p} & {m}_{12}^{p} \\ {m}_{20}^{p} & {m}_{21}^{p} & {m}_{22}^{p}\end{matrix} ) ( \begin{matrix}Δ x \\ Δ y \\ Δ z\end{matrix} ) + ( \begin{matrix}{m}_{00}^{p} & {m}_{01}^{p} & {m}_{02}^{p} \\ {m}_{10}^{p} & {m}_{11}^{p} & {m}_{12}^{p} \\ {m}_{20}^{p} & {m}_{21}^{p} & {m}_{22}^{p}\end{matrix} ) ( \begin{matrix}{r}_{00} & {r}_{01} & {r}_{02} \\ {r}_{10} & {r}_{11} & {r}_{12} \\ {r}_{20} & {r}_{21} & {r}_{22}\end{matrix} ) ( \begin{matrix}{x}_{\_ b 1}^{p} \\ {y}_{\_ b 1}^{p} \\ {z}_{\_ b 1}^{p}\end{matrix} ) \\ ( \begin{matrix}{x}_{\_ t}^{2} \\ {y}_{\_ t}^{2} \\ {z}_{\_ t}^{2}\end{matrix} ) = ( \begin{matrix}{x}_{p}^{′} \\ {y}_{p}^{′} \\ {z}_{p}^{′}\end{matrix} ) ( \begin{matrix}{m}_{00}^{p} & {m}_{01}^{p} & {m}_{02}^{p} \\ {m}_{10}^{p} & {m}_{11}^{p} & {m}_{12}^{p} \\ {m}_{20}^{p} & {m}_{21}^{p} & {m}_{22}^{p}\end{matrix} ) ( \begin{matrix}Δ x \\ Δ y \\ Δ z\end{matrix} ) + ( \begin{matrix}{m}_{00}^{p} & {m}_{01}^{p} & {m}_{02}^{p} \\ {m}_{10}^{p} & {m}_{11}^{p} & {m}_{12}^{p} \\ {m}_{20}^{p} & {m}_{21}^{p} & {m}_{22}^{p}\end{matrix} ) ( \begin{matrix}{r}_{00} & {r}_{01} & {r}_{02} \\ {r}_{10} & {r}_{11} & {r}_{12} \\ {r}_{20} & {r}_{21} & {r}_{22}\end{matrix} ) ( \begin{matrix}{x}_{\_ b 2}^{p} \\ {y}_{\_ b 2}^{p} \\ {z}_{\_ b 2}^{p}\end{matrix} ) \\ ………… \\ ( \begin{matrix}{x}_{\_ t}^{N} \\ {y}_{\_ t}^{N} \\ {z}_{\_ t}^{N}\end{matrix} ) = ( \begin{matrix}{x}_{p}^{′} \\ {y}_{p}^{′} \\ {z}_{p}^{′}\end{matrix} ) ( \begin{matrix}{m}_{00}^{p} & {m}_{01}^{p} & {m}_{02}^{p} \\ {m}_{10}^{p} & {m}_{11}^{p} & {m}_{12}^{p} \\ {m}_{20}^{p} & {m}_{21}^{p} & {m}_{22}^{p}\end{matrix} ) ( \begin{matrix}Δ x \\ Δ y \\ Δ z\end{matrix} ) + ( \begin{matrix}{m}_{00}^{p} & {m}_{01}^{p} & {m}_{02}^{p} \\ {m}_{10}^{p} & {m}_{11}^{p} & {m}_{12}^{p} \\ {m}_{20}^{p} & {m}_{21}^{p} & {m}_{22}^{p}\end{matrix} ) ( \begin{matrix}{r}_{00} & {r}_{01} & {r}_{02} \\ {r}_{10} & {r}_{11} & {r}_{12} \\ {r}_{20} & {r}_{21} & {r}_{22}\end{matrix} ) ( \begin{matrix}{x}_{\_ bN}^{p} \\ {y}_{\_ bN}^{p} \\ {z}_{\_ bN}^{p}\end{matrix} ) \\ ………… \qquad ( 4 )$$

wherein: p represents p^{th} time to get and record the center position data of N marker/markers at different p times position, and p=5 if N=1, p=3 if N=2 or N=3, and p=2 if N≥4; and (x__{b}, y__{b}, Z__{b}) represents a known center position data of the marker with respect to the coordinate system of the vision measuring system; and
(x′, y′, z′) represents a known position data of the tracking tool; and a matrix

$$( \begin{matrix}{m}_{00} & {m}_{01} & {m}_{02} \\ {m}_{10} & {m}_{11} & {m}_{12} \\ {m}_{20} & {m}_{21} & {m}_{22}\end{matrix} )$$

is known for direction data of the tracking tool; and (xp_bN, yp_bN, zp_bN) represents a center position data of Nth marker on pth position's recording; and (xp′, yp′, zp′) represents a position data of the tracking tool on pth position's recording; and a matrix:

$$( \begin{matrix}{m}_{00}^{p} & {m}_{01}^{p} & {m}_{02}^{p} \\ {m}_{10}^{p} & {m}_{11}^{p} & {m}_{12}^{p} \\ {m}_{20}^{p} & {m}_{21}^{p} & {m}_{22}^{p}\end{matrix} )$$

is known for direction data of the tracking tool on p^{th} position's recording; and (x^{N}_t, y^{N}_t, z^{N}_t) represents a center position of N^{th} marker with respect to the tracking assembly's coordinate system; and (Δx, Δy, Δz) represents the position calibration offset between the coordinate system of the vision measuring system and the tracking tool; and

$$( \begin{matrix}{r}_{00} & {r}_{01} & {r}_{02} \\ {r}_{10} & {r}_{11} & {r}_{12} \\ {r}_{20} & {r}_{21} & {r}_{22}\end{matrix} )$$

represents the directional calibration relationship between the coordinate system of the vision measuring system and the tracking tool.

In step c) the solving, based on the at least p groups of relative center position data of N marker/markers, nonhomogeneous linear equations to thereby obtain calibration relationship between the designated coordinate system of the vision measuring system and the tracking tool, wherein the nonhomogeneous linear equations are derived from the relationship between a center position of spherical marker with respect to the designated coordinate system of the vision measuring system and that position with respect to the coordinate system of the tracking assembly comprises:

solving the formula (4) of at least N×3×p equations in at least p×N equation groups to thereby obtain: the position offset: (Δx, Δy, Δz); and the matrix of direction calibration:

$$( \begin{matrix}{r}_{00} & {r}_{01} & {r}_{02} \\ {r}_{10} & {r}_{11} & {r}_{12} \\ {r}_{20} & {r}_{21} & {r}_{22}\end{matrix} ) .$$

In some embodiments, the marker for each target comprises a first portion and a second portion; and the first portion has a shape of a sphere and is substantially at a core center of the spherical marker; and the second portion is at an outer layer of the spherical marker and is arranged such that a core center of the second portion also substantially coincides with the core center of the first portion; and the first portion and the second portion have different compositions capable of generating a relatively either weak or strong signal compared each other by a diagnostic imaging scanner, as such, in the scanned imaging, the image position of center of the first portion of the marker can be determined and measured easily and accurately with distinguishingly displayed spot.

In some embodiments, the at least one target is the at least four targets and the method of measuring target's position further comprising:
a) reconstituting, based on the at least four markers of targets, a group position data for an area comprising the at least four targets with respect to a reference coordinate system of the tracking assembly, wherein: the at least four target positions are not coplanar in three-dimensional space; and each target has a rigid fixed position relatively to each other; and the origin and direction of the reference coordinate system of the tracking assembly is arranged at a rigid fixed position and direction relatively to the group positions of the at least four markers of targets;
b) scanning an object for navigation and the group of the at least four markers of targets together via an imaging scanner, to obtain a group of imaging position data of the at least four markers of targets, wherein the relative position and direction among the object for navigation, the origin and direction of the reference coordinate system of the tracking assembly and each of the at least four markers of targets are rigid fixed each other;
c) calculating, based on the two groups of position data in imaging world and that in physical world, the transformation of positions and directions between the physical word and the imaging world, which is used for navigation regarding the object, under the condition that the relative position and direction between the object and the origin and direction of the reference coordinate system of the tracking assembly are rigid fixed and unchanged from the above scanning step b).

In some embodiments, the tracking assembly comprises a transmitter configured to generate an electromagnetic field; and the tracking tool comprises a sensing coil configured to produce an induced voltage in the electromagnetic field; and the tracking assembly further comprises an electronics unit, is coupled to the sensing coil and the transmitter and is configured to calculate the position and orientation data of the tracking tool based on the induced voltage produced in the sensing coil; and the reference coordinate system of the tracking assembly is bases on a tracking tool of six-degree of position and direction.

### BRIEF DESCRIPTION OF THE DRAWINGS

FIG. 1 is a schematic diagram of using a register pen to determine a position of a marker according to a conventional technology;

FIG. 2 illustrates a marker position measuring system according to some embodiments of the disclosure;

FIG. 3A illustrates a marker with a shape of a sphere according to the first embodiment of the disclosure;

FIG. 3B illustrates a marker with a shape of a hemi-sphere according to the second embodiment of the disclosure;

FIG. 3C illustrates a marker with a shape of a convex surface according to the third embodiment of the disclosure;

FIG. 4A illustrates a cross-sectional view of a marker according to one embodiment of the disclosure;

FIG. 4B illustrates a cross-sectional view of a marker according to another embodiment of the disclosure;

FIG. 5A is a cross-section view of a first member of a tracking assembly configured to measure a marker's position in a contacting manner according to some embodiments of the disclosure;

FIG. 5B is a perspective view of the measuring head of the tracking assembly as shown in FIG. 5A;

FIG. 6 illustrates a schematic diagram of a marker position measuring system configured to measure a marker's position in a contacting manner according to one embodiment of the disclosure;

FIG. 7 illustrates a flow chart of a method using the first embodiment of the marker position measuring system for determining a 3D position of a dot to be measured in a space in a contacting manner according to some embodiments of the disclosure;

FIG. 8 illustrates a schematic diagram of a first member of a tracking assembly configured to measure a marker's position in a non-contacting manner according to some embodiments of the disclosure;

FIG. 9 illustrates a schematic diagram of the principle of utilizing a binocular vision measuring system to obtain the position information of an object;

FIG. 10 illustrates a schematic diagram of a marker position measuring system configured to measure a marker's position in a non-contacting manner according to one embodiment of the disclosure;

FIG. 11 is a flowchart of a method using the second embodiment of the marker position measuring system for determining a 3D position designated to be measured in a space in a non-contacting manner according to some embodiments of the disclosure; and

FIG. 12 is a flowchart of a method for obtaining the calibration relationship between the designated coordinate system of the vision measuring system and the tracking tool according to some embodiment of the disclosure.

### DETAILED DESCRIPTION

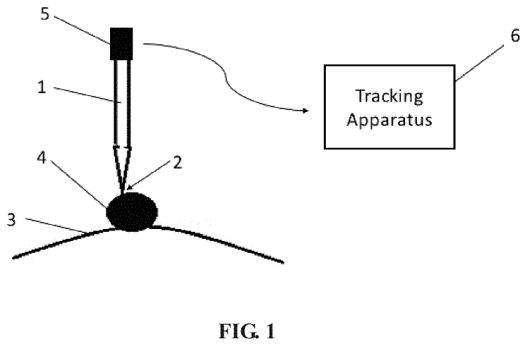

FIG. 1 illustrates a schematic diagram of using a register pen to determine the position of a marker according to a conventional technology. As shown in the figure, a register pen 1 having a sharp tip 2 (indicated by an arrow) is configured to point to, and touch, a marker 4 (shown as a dot in FIG. 1) on skin 3 of a patient with its sharp tip 2.

The register pen 1 is typically equipped with a tracking sensor or tool 5 fixedly attached onto the register pen 1. The tracking sensor or tool 5 is coupled with a tracking apparatus 6. The tracking apparatus 6 is configured to acquire signals (infrared, optical, electromagnetic, ultrasound etc.) from the tracking sensor or tool 5 and is further configured to deduce, calculate and then obtain the position and orientation parameters of the tracking sensor or tool 5.

Based on the position and orientation parameters of the tracking tool/sensor 5 and the positional relationship between the tracking fool/sensor 5 and the tip 2 of the register pen 1, the tracking apparatus 6 is further configured to calculate a position of the tip 2 of the register pen 1 to thereby obtain a position of the marker 4 (i.e., the position of the tip of the register pen 1 is substantially a surrogate of the position of the marker 4).

As illustrated in FIG. 1, the marker 4 on the skin 3 is not an ideal dot, yet with a finite size, and thus the marker 4 can have many points on a physical surface thereof. As such, if the tip 2 of the register pen 1 points to and touches a different position (point) on the marker 4 within its physical surface, the measurement by the register pen 1 can give a different value depending on the actual position of the point on the surface of the marker 4, causing an issue of limited accuracy in determining the marker 4's real position.

To solve the aforementioned inaccuracies associated with the current technologies, one approach is to make the markers as smaller as possible, meanwhile making the markers large enough to be perceived via CT/MRI images within its image resolution limit. Instead of using a sharp-tip register pen and tiny markers, various embodiments of the present disclosure provide other approaches. Described below are some embodiments of method for tracking targets, such as measuring markers in a surgical navigation system.

In some embodiments, a system for measuring a three-dimensional (3D) position of a target is provided. The target can be, for example, a marker, and the system can be referred to as a marker position measuring system. In some embodiments, the target can be a marker drawn or printed on a patient's sky. In some other embodiments, the target can be a physical object, such as a sticker, a pin, a bead, etc., that is to be tracked. The target can be removably or permanently affixed to an object, such as a patient, for measurements/position tracking.

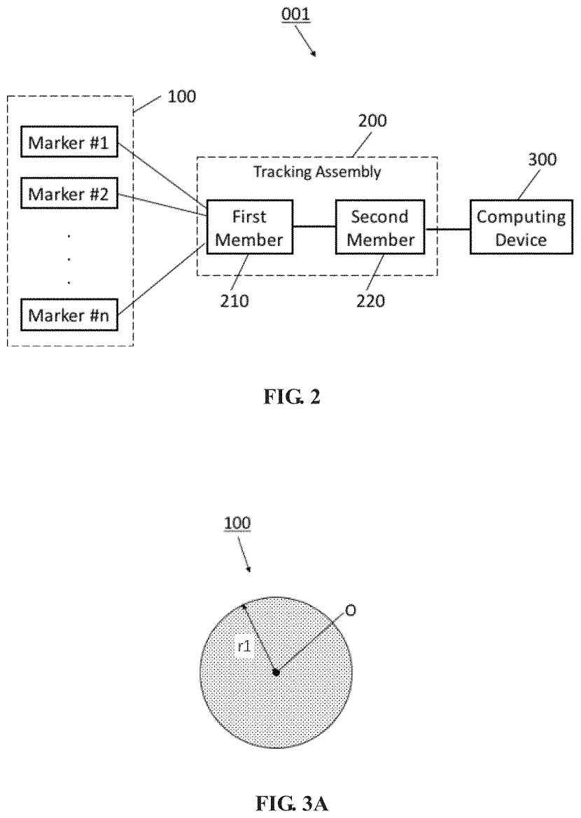

FIG. 2 illustrates a marker position measuring system according to some embodiments of the disclosure. As shown in FIG. 2, the marker position measuring system 001 is configured to measure a position of at least one marker 100, and includes a tracking assembly 200 and a computing device 300. The computing device 300 can include one or more processors or processing circuits, and non-transitory memory having software program product (such as instructions) stored thereon which, when executed by the computing device 300, can realize algorithms and methods as described below, and/or steps to compute, analyze, and display outputs to a user.

The at least one marker 100 (illustrated as marker #1, #2, . . . , and #n in FIG. 2, where n is an integer greater than zero) is respectively disposed at a different position about a patient. The tracking assembly 200 is coupled to each of the at least one marker 100 and to the computing device 300. The computing device 300 is configured to calculate a 3D position for each of the at least one marker 100.

The tracking assembly 200 further comprises a first member 210 and a second member 220, as illustrated in FIG. 2. The first member 210 is coupled to each of the at least one marker 100, and is configured to obtain relative position data of each of the at least one marker 100 with respect to the first member 210.

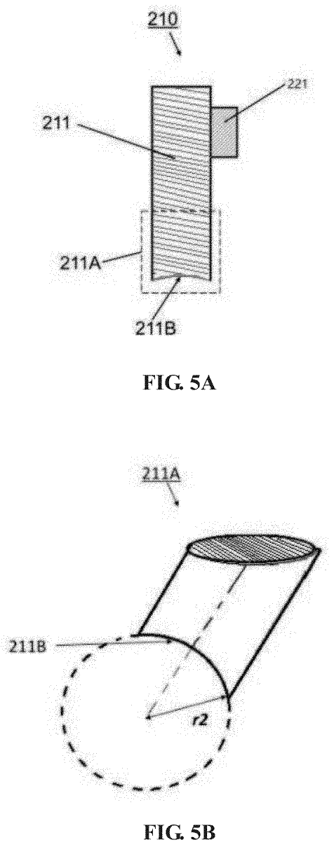

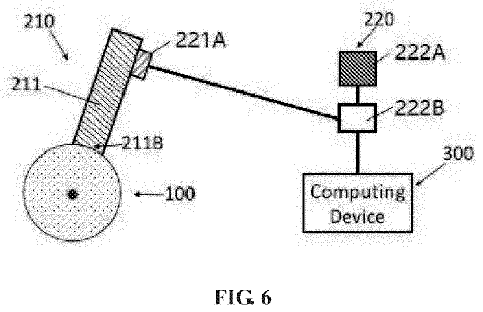

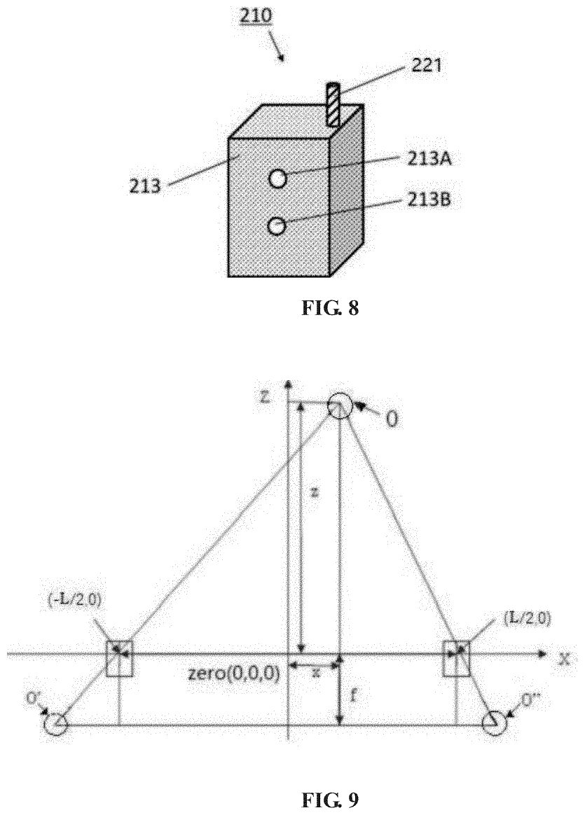

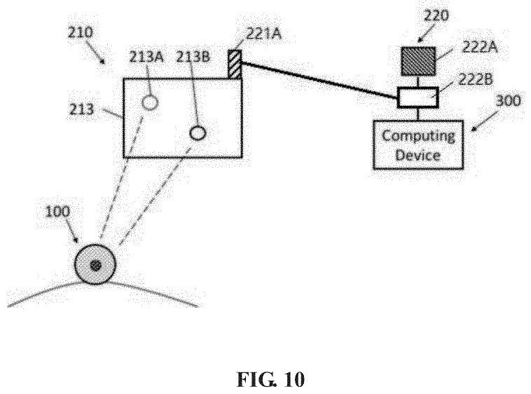

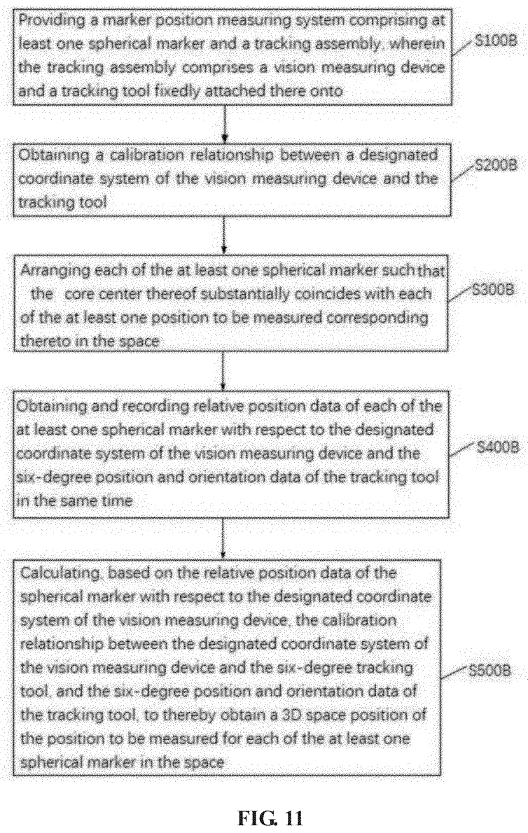

The second member 220 is coupled to the first member 210, and is configured to obtain position and orientation data (e.g., six-degree position and orientation data) of the first member 210 by means of, for example, a tracking tool fixedly attached onto the first member 210 (not shown in FIG. 2, but illustrated in FIG. 5A, FIG. 6, FIG. 8 and FIG. 10 that follow).

The computing device 300 is configured to determine a 3D position of each of the at least one marker 100, based on the relative position data of each of the at least one marker 100 with respect to the first member 210, and the position and orientation data of the first member 210.

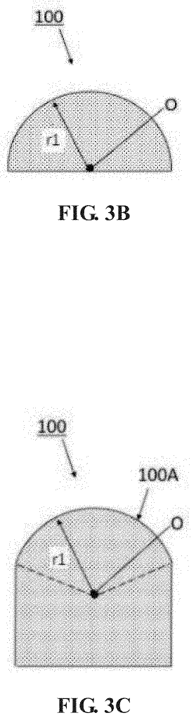

According to some embodiments, in the marker position measuring system 001, each of the at least one marker 100 takes a shape of a sphere, and thus is substantially a spherical marker, as illustrated in FIG. 3A. The tracking assembly 200 can be specifically configured to measure position data of a geometric center (or a core center) O of each spherical marker 100, based on which a 3D position of the core center O of each marker can be determined.

It is noted that the shape of each marker 100 is not limited to a sphere. According to different embodiments, each marker 100 can, for example, take a shape of a hemi-sphere (as illustrated in FIG. 3B), or take a partial sphere (not shown), or take a special shape comprising a convex surface 100A that is part of a sphere (as illustrated in FIG. 3C). In some other embodiments, non-spherical shapes can be employed for the marker 100. In an example, the marker 100 can have an oval shape. In another example, the marker can have a shape of a cube, a cone, a rectangle, etc.

Regardless of different shapes of a marker 100, the marker position measuring system 001 according to some embodiments of the present disclosure can be utilized to measure a 3D position of the core center O of the marker 100. By disposing a marker 100 at a specific position corresponding a dot X in the 3D space, the 3D position of the dot can be obtained by measuring the 3D position of the core center O of the marker 100, where the core center O can be regarded to represent the dot X. The dot X can be, for example, a position on a patent's skin, skull, or organ, of which once the position is accurately measured using the marker position measuring system 001, precision operations can be made with reference to the position, employing the surgical navigation system.

In some embodiments, the dot X can move around, for example when a patient breathes causing skin movement around the chest area. In such cases dynamic position measuring can be performed in real time using the marker position measuring system 001 disclosed herein.

In some embodiments, the position measurements or tracking are not limited to medical applications, and can be employed in other areas of applications such as geology, architecture, biological research, etc.

As such, compared with a conventional method to measure a 3D position of a dot using a register pen to measure the 3D position of a marker disposed at the dot as illustrated in FIG. 1, the marker position measuring system 001 disclosed herein substantially transforms a dot to be measured into a core center of a marker having a convex surface (e.g., part or whole of a sphere), and through the measuring of position data of the core center, the 3D position of the dot can be obtained with a relatively high accuracy due to the effective solving of the issue of varied positions of the register pen touching on the marker surface as illustrated in FIG. 1.

It is noted that the convex surface of each marker substantially serves as a measuring surface for the tracking assembly to measure position data of the core center of each marker according to some embodiments, and will be described below in detail.

Depending on actual applications, each marker 100 can comprise a first portion 110 and a second portion 120 according to some embodiments of the disclosure. The first portion 110 and the second portion 120 are arranged at a core center and an outer layer of the marker, respectively.

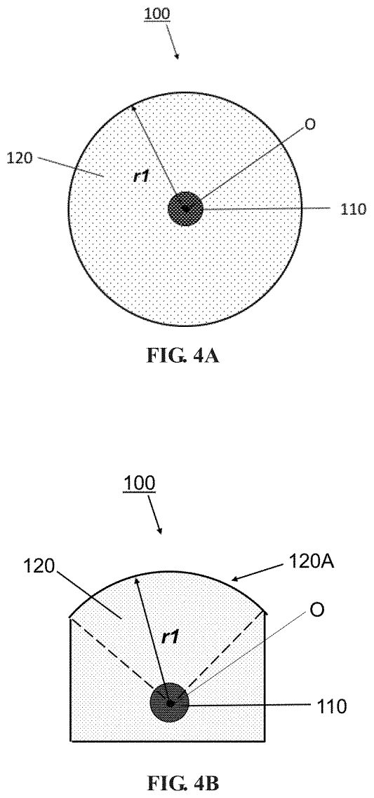

FIG. 4A is a cross-sectional view of one marker 100 according to some embodiments of the present disclosure. As illustrated, the marker 100 is substantially a spherical marker having a radius of r1. The first portion 110 has a shape of a small sphere and is substantially at a core center of the spherical marker 100 (i.e., a core center of the first portion 110 substantially coincides with the core center of the spherical marker 100). The second portion 120 is at an outer layer of the spherical marker 100, and is arranged such that a core center of the second portion 120 also substantially coincides with the core center of the first portion 110.

Furthermore, the first portion 110 and the second portion 120 are configured to have a different composition allowing for differential perception by a diagnostic scanner, such as a CT scanner or an MRI scanner. The first portion 110 is further configured to be as small as possible to allow a better accuracy of position measurement with CT/MRI, yet to be large enough to be perceived in images by the diagnostic scanner (i.e., CT/MRI images) within a resolution limit for the images. The second portion 120 of the marker 100 is configured to be sufficiently rigid, allowing secure embedding and attachment of the first portion 110 therein.

According to some embodiments of the marker position measuring system 001 compatible to a CT image application, the first portion 110 of the marker 100 comprises a small sphere having a composition of a CT signal strong material, such as a metal material, and the second portion 120 of the marker 100 substantially comprises a CT signal weak material, such as a non-metal material (e.g. a plastic). As such, in a CT image for measurement, only the first portion 110 at the geometric center of the spherical marker 100 can be distinguishingly displayed as a bright spot.

According to some other embodiments of the marker position measuring system 001 compatible to a CT image application, the first portion 110 of the marker 100 comprises a small sphere having a composition of a CT signal weak material, such as a plastic material, and the second portion 120 of the marker 100 substantially comprises a CT signal strong material, such as a metal material. As such, in a CT image for measurement, only the first portion 110 at the geometric center of the spherical marker 100 can be distinguishingly displayed as a dark spot.

According to some embodiments of the marker position measuring system 001 compatible to an MRI application, the first portion 110 of the marker 100 comprises a small sphere having a composition of an MRI signal strong material, such as a liquid material, and the second portion 120 of the marker 100 substantially comprises an MRI signal poor material. As such, in an MRI image for measurement, only the first portion 110 at the geometric center of the spherical marker 100 can thus be distinguishingly displayed as a bright spot.

According to some other embodiments of the marker position measuring system 001 compatible to an MRI application, the first portion 110 of the marker 100 comprises a small sphere having a composition of an MRI signal weak material, such as a gold material, and the second portion 120 of the marker 100 substantially comprises an MRI signal strong material. As such, in an MRI image for measurement, only the first portion 110 at the geometric center of the spherical marker 100 can thus be distinguishingly displayed as a dark spot.

In other words, the first portion 110 and the second portion 120 of the marker 100 have different compositions capable of generating a relatively either weak or strong signal compared to each other by a diagnostic imaging scanner, as such, in the scanned images, only the first portion at the geometric center of spherical marker 100 can be distinguishingly displayed as an either bright or dark spot and be measured easily and accurately.

FIG. 4B shows a cross-sectional view of a marker 100 having a non-spherical shape according to some other embodiment of the disclosure. Similar to the embodiments of the marker as illustrated in FIG. 4A, the marker 100 also comprises a first portion 110 having a shape of a small sphere and is embedded in the second portion 120. The second portion 120 comprises a convex surface 120A (as indicated by the arrow in FIG. 4B), configured to be a portion of a sphere having a radius of r1. It is configured such that the first portion 110 is substantially at a core center of the convex surface 120A of the second portion 120 (i.e., the core center of the convex surface 120A of the second portion 120 is substantially a core center of a sphere to which the convex surface 120A belongs).

Similar to the embodiments as mentioned above and illustrated in FIG. 4A, the first portion 110 and the second portion 120 of the marker 100 as illustrated in FIG. 4B can respectively comprise a composition of a CT signal strong/weak material and a CT signal poor/strong material, or an MRI signal strong/weak material and an MR signal poor/strong material, as such, in the scanned images, only the first portion at the geometric center of spherical marker 100 can be distinguishingly displayed as an either bright or dark spot and be measured easily and accurately, depending on the actual applications in CT scanning or MRI scanning.

In addition to the arrangement of the first portion 110 and the second portion 120 in the marker 100 as illustrated in FIGS. 4A and 4B, other arrangements are also possible. For example, the first portion 110 can be on a surface of the second portion 120, as long as the first portion, still as a small sphere, is substantially at a core center of a convex surface 120A of the second portion 120.

According to some embodiments, the convex surface 120A is configured, according to some embodiments of the disclosure, as the contact surface for the measuring head 211A of the measuring piece 211 in the first member 210 of the tracking assembly 200 as illustrated in FIGS. 5A, 5B, and 6. According to some other embodiments, the convex surface 120A is configured to serve as a surface to be observed by the binocular vision measuring system in the first member 210 of the tracking assembly 200 as illustrated in FIG. 10. As such, the convex surface 120A is substantially a measuring surface of the marker 100 in the marker position measuring system disclosed herein.

Herein, by configuring a first portion of a CT/MRI signal-strong composition at a core center of a marker having an outer convex surface, the physical world 3D position of the core center of the marker can be accurately calculated by means of the marker position measuring system, meanwhile the image position of the first portion of the marker in a CT/MRI image can also be determined easily and accurately with distinguishingly displayed bright spot.

Similarly, by configuring a first portion of a CT/MRI signal weak composition at a core center of a marker having an outer convex surface, the physical world 3D position of the core center of the marker can be accurately calculated by means of the marker position measuring system, meanwhile the image position of the first portion of the marker in a CT/MRI image can also be determined easily and accurately with distinguishingly displayed dark spot.

Depending on whether or not the first member 210 of the tracking assembly 200 obtains the relative position data of each of the at least one marker 100 with respect to the first member 210 in a contacting manner or in a non-contacting manner, there are two different embodiments of the marker position measuring system 001: the first embodiment of the marker position measuring system 001 and the second embodiment of the marker position measuring system 001 described respectively below.

In the first embodiment of the marker position measuring system 001, the first member 210 of the tracking assembly 200 is configured to obtain the relative position data of each of the at least one marker 100 with respect to the first member 210 in a contacting manner. Each of the at least one marker 100 is configured to comprise a convex surface which is part or a whole of a sphere, and thus can be a spherical marker as illustrated in FIG. 3A, a hemispherical marker as illustrated in FIG. 3B, or a marker having a convex surface as illustrated in FIG. 3C, or other possibilities.

FIG. 5A illustrates a cross-sectional view of a first member 210 of a tracking assembly 200 according to some embodiments of the disclosure. As shown in the figure, the first member 210 substantially comprises a measuring piece 211. A tracking tool 221 is fixedly attached onto the measuring piece 211. The tracking tool 221 is considered as a component of the second member 220 of the tracking assembly 200, which is for obtaining the position and orientation parameters of the first member 210. The measuring piece 211 comprises a measuring head 211A (as indicated by the box with dotted lines), which is provided with a concave surface 211B. The tracking tool 221 can be a tracking sensor, such as an electromagnetic tracking sensor according to some embodiments of the disclosure, or can be balls, such as infrared tracking tools according to some other embodiments of the disclosure.

Further as illustrated by FIG. 5B, the concave surface 211B on the measuring head 211A of the first member 210 is substantially part or portion of a surface of a sphere (i.e. comprises a spherical surface as illustrated by the circle with a dotted line in FIG. 5B), configured such that a radius r2 thereof is substantially the same as the radius r1 of the sphere of the convex surface in each marker 100 as illustrated in any of FIGS. 3A, 3B, 3C, 4A, or 4B. As such, the concave surface 211B on the measuring head 211A of the first member 210 of the tracking assembly 200 can matchingly fit with the convex surface on the marker 100. The concave surface 211B is substantially a measuring surface for the measuring piece 211.

As such, no matter where the measuring head 211A of the first member 210 of the tracking assembly 200 is placed onto the measuring surface (i.e., the convex surface) of a marker 100 corresponding in shape and size to the concave surface 211B of the measuring head 211A, the contact between the measuring head 211A of the measuring piece 211 and the marker 100 is substantially fit and secure.

On the other hand, because the convex surface on the marker 100 is substantially a part or a whole of a sphere, which has a fixed core center (i.e., the geometric center of the sphere), the relative position data of each marker 100 with respect to the first member 210 of the tracking assembly 200 can be relatively more accurate, in turn allowing the subsequent calculation of the 3D position of each marker 100 to be relatively more accurate.

As such, the issue of inaccuracy associated with the less-accurate positioning (e.g., varied positions) of a conventional register pen that is commonly utilized during measurement of a position of a marker during registering (as illustrated in FIG. 1) can be effectively avoided.

Depending on different ways for the second member 220 to obtain the position and orientation data of the first member 210, there can be multiple embodiments for configuring the first member 210 and the second member 220 in the tracking assembly 200.

According to some embodiments of applying electromagnetic tracking assembly as illustrated in FIG. 6, the second member 220 of the tracking assembly 200 comprises a transmitter 222A configured to generate an electromagnetic field, a tracking tool 221A and an electronics unit 222B. The tracking tool 221A is fixedly attached onto the measuring piece 211 of the first member 210.

The tracking tool 221A includes, for example, a sensing coil, and is configured to produce an induced voltage in the electromagnetic field generated by the transmitter 222A. The electronics unit 222B is coupled to the sensor 221A to obtain the induced voltage produced in the sensor 221A and is coupled to the computing device 300 wiredly or wirelessly, to calculate position and orientation data of the first member 210 (or more specifically, the position and orientation data of the sensor 221A).

According to some embodiments of applying infrared tracking assembly, the second member 220 of the tracking assembly 200 can comprise a camera 222A configured to emit infrared light and to take infrared photos, a tracking tool 221A, and an electronics unit 222B. The tracking tool 221A includes, for example, balls to reflect infrared light.

The computing device 300 can further combine the position and orientation data of the first member 210 (or more specifically, of the tracking tool 221A) of the tracking assembly 200 and the relative position data of each of the at least one marker 100 with respect to the first member 210 (or more specifically, with respect to the tracking tool 221A) of the tracking assembly 200 to thereby deduce the position of each of the at least one marker 100.

It is noted that the relative position data of each of the at least one marker 100 with respect to the first member 210 of the tracking assembly 200 can be considered as position data of each marker 100 in a relative coordinate system with the first member 210 as a reference point, and that the position and orientation data of the first member 210 of the tracking assembly 200 can be considered as position and orientation data in an absolute coordinate system with a reference coordinate system having a fixed position and direction in the space (e.g., the transmitter 222A in the embodiment as shown in FIG. 6, or another tracking tool as reference bases of position and direction).

Therefore, by combining the position and orientation data of the first member 210 with the relative position data of each of the at least one marker 100 with respect to the first member 210, the 3D position of each of the at least one marker 100 in the absolute coordinate system can be deduced.

It is noted that the above embodiments as shown in FIG. 6 serve as an illustrating example only and shall not be regarded as a limitation to the scope of the disclosure. There can be other embodiments as well.

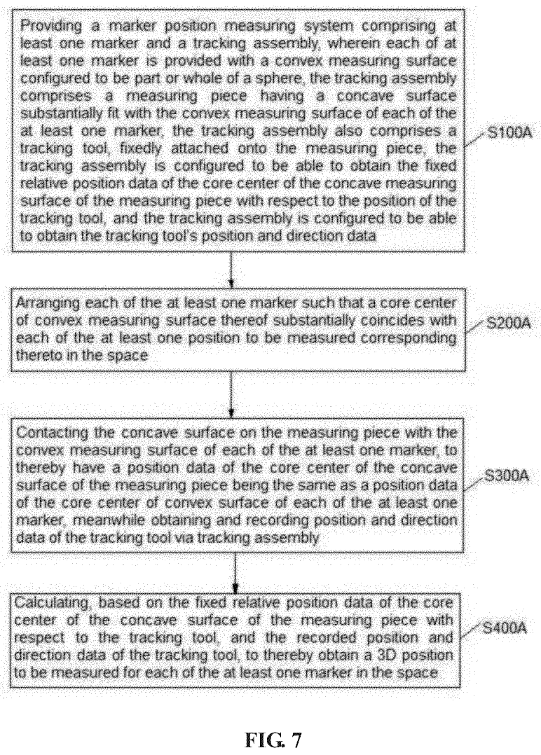

In the following, a method of using the abovementioned first embodiment of the marker position measuring system for determining a 3D position of a dot to be measured in a space in a contacting manner is provided. Specifically, as illustrated by the flow chart in FIG. 7, the method comprises the following steps.

S100A: providing a marker position measuring system comprising at least one marker and a tracking assembly, wherein each of the at least one marker is provided with a convex measuring surface configured to be part or whole of a sphere, the tracking assembly comprises a measuring piece having a concave surface substantially fit with the convex measuring surface of each of the at least one marker, the tracking assembly also comprises a tracking tool, fixedly attached onto the measuring piece, the tracking assembly is configured to be able to obtain the fixed relative position data of the core center of the concave measuring surface of the measuring piece with respect to the position of the tracking tool, and the tracking assembly is configured to be able to obtain the tracking tool's position and direction data;

S200A: arranging each of the at least one marker such that a core center of convex measuring surface thereof substantially coincides with each of the at least one position to be measured corresponding thereto in the space;

S300A: contacting the concave surface on the measuring piece with the convex measuring surface of each of the at least one marker, to thereby have a position data of the core center of the concave surface of the measuring piece being the same as a position data of the core center of convex surface of each of the at least one marker, meanwhile obtaining and recording position and direction data of the tracking tool via tracking assembly;

S400A: calculating, based on the fixed relative position data of the core center of the concave surface of the measuring piece with respect to the tracking tool, and the recorded position and direction data of the tracking tool, to thereby obtain a 3D position to be measured for each of the at least one marker in the space.

Specifically, the marker and the tracking assembly can be based on any of the embodiments as described and illustrated above. In one specific application, the position to be measured is on skin of a patient, and the marker can comprise a first portion of a CT/MRI signal-strong composition and a second portion of a CT/MR signal-poor composition, respectively arranged at the core center and elsewhere of the marker.

The maker can also comprise a first portion of a CT/MRI signal-poor composition and a second portion of a CT/MRI signal-strong composition, respectively arranged at the core center and elsewhere of the marker. With such a way, the image position of the first portion of the marker in a CT/MRI image can be determined easily and accurately with distinguishingly displayed spot. The method thereby can be used to positionally match the 3D position of the marker in physical space with that in CT/MRI scan images.

It is noted that in some applications, a bigger area, not just one individual position of dot, needs measurement for determining the spatial position and conformation thereof. Without considering its inside structure, the area to be measured can be regarded to include a plurality of dots, configured such that each dot is on a different position on the area, and the plurality of dots together can sufficiently represent the area. By measuring the position of each of the plurality of dots on the area, the spatial position and conformation of the area can be approximately determined.

For this purpose, this first embodiment of the marker position measuring system as described above can be utilized for determining a spatial position and conformation of an area to be measured in a space.

It is noted that it is possible that this first embodiment of the marker position measuring system comprises only one marker, instead of a plurality of markers, and this marker can be repeatedly used to measure a 3D position of each of the set of dots with designated positions on the area.

In the second embodiment of the marker position measuring system 001, the first member 210 of the tracking assembly 200 is configured to obtain relative position data of each of the at least one marker 100 with respect to the first member 210 in a non-contacting manner.

Each of the at least one marker 100 comprises a spherical marker as illustrated in FIGS. 3A, 3B or 3C. As illustrated in FIG. 8, the first member 210 of the tracking assembly 200 substantially comprises a vision measuring system 213 (for example, having two cameras 213A and 213B) and a tracking tool 221 which is fixedly attached with the vision measuring system 213. Similar to the first embodiment of the marker position measuring system, the tracking tool 221 can also be a tracking sensor (e.g., an electromagnetic tracking sensor, or infrared tracking balls), depending on different embodiments of the disclosure.

Further as illustrated in the embodiments in FIG. 10, the first member 210 of the tracking assembly 200 is arranged at a distance from a marker 100, arranged such that the image sensing assembly of the vison measuring system is facing the marker 100. In this embodiment in FIG. 10, the image sensing assembly of the vison measuring system includes two cameras 213A and 213B. The vision measuring system 213 is configured to obtain relative position data of each of the at least one marker 100 (or more specifically the geometric core center of each spherical marker 100) with respect to some reference coordinate system of the vision measuring system 213 of the first member 210 of the tracking assembly 200.

The vision measuring system can be configured to have different number of cameras. A binocular device is an example. The principle of utilizing the two cameras 213A and 213B of the vision measuring system 213 to obtain the position information of an object O is illustrated in FIG. 9. As shown, the object O has two images O′ and O″ in the two cameras respectively. The reference f represents focal length of the two cameras, and the reference L represents a distance between the two cameras. With a computation, the coordinates x, y, z of object O can be obtained with respect to the designated coordinate system with indicated zero (0,0,0) point, which is substantially at the middle of the two cameras.

As long as the vision measuring system 213 can perceive the spherical marker 100, the vision measuring system 213 can calculate the relative position of the geometric core center of the spherical marker 100 (i.e., the position of the geometric core center of the spherical marker 100 in the relative coordinate system with respect to the system 213 of the first member 210 of the tracking assembly 200). It is noted that a relative coordinate system having its zero point arranged at a position rather than the middle of two cameras can also be applied (for example, a relative coordinate system having its zero point arranged at one camera), and there are no limitations herein.

Herein in the marker position measuring system disclosed, each of the vision measuring system 213, the tracking tool 221, or the other second member 220 can be wiredly or wirelessly connected with other modules of the system, such as the computing device 300.

Depending on different ways for the second member 220 to obtain the position and orientation data of the first member 210, there can be multiple embodiments for configuring the first member 210 and the second member 220 in the tracking assembly 200.

According to one embodiment of applying electromagnetic tracking assembly as illustrated in FIG. 10, the second member 220 of the tracking assembly 200 comprises a transmitter 222A configured to generate an electromagnetic field, a tracking tool 221A and an electronics unit 222B. The tracking tool 221A fixedly attached onto the binocular vision measuring system 213 of the first member 210 comprises a sensor. The sensor 221A contains a sensing coil and thus can produce an induced voltage in the electromagnetic field generated by the transmitter 222A. The electronics unit 222B is coupled to the sensor 221A to obtain the induced voltage produced in the sensor and is coupled to the computing device 300 wiredly or wirelessly, to calculate position and orientation data of the tracking tool 221A, thus of the vision measuring system 213 of the first member 210.

The computing device 300 can further combine the position and orientation data of the first member 210 with the relative position data of core center of each of the at least one marker 100 with respect to the first member 210 to thereby deduce the 3D position of core center of each of the at least one marker 100.

It is noted that in the above embodiments as shown in FIG. 10, since the tracking tool 221A is fixedly attached onto the vision measuring system 213, the geometric relationship between the tracking tool 221A and the rigid vision measuring system 213 is constant. As such, there is a translational relationship between the zero point of the designated coordinate system of the vision measuring system 213 and the position of the tracking tool 221A. There is also a rotational relationship between coordinate axis of the designated coordinate system of the vision measuring system 213 and the directions of the tracking tool 221A. Those constant relationships can be obtained by measurement/calibration.

In the following, a method of using this above mentioned second embodiment of the marker position measuring system for determining a 3D position designated to be measured in a space in a non-contacting manner is provided.

The method is substantially based on: 1. the relative position data of the spherical marker with respect to the designated coordinate system of the vision measuring system 213 (i.e., the 3D space position of the core center of the spherical marker with respect to the designated coordinate system of the vision measuring system); 2. the calibration relationship between a designated coordinate system of the vision measuring system 213 and the tracking tool 221A (e.g., the sensor 221A in FIG. 10); 3. the six-degree position and orientation data of the tracking tool 221A for calculating the 3D position of the spherical marker (or more specifically, of the core center of the spherical marker, which substantially represents the designated position to be measured in the space).

Specifically, as illustrated by the flow hart in FIG. 11, the method comprises the following steps:

S100B: providing a marker position measuring system comprising at least one spherical marker and a tracking assembly, wherein the tracking assembly comprises a vision measuring system and a tracking tool fixedly attached there onto;

S200B: obtaining a calibration relationship between a designated coordinate system of the vision measuring system and the tracking tool;

S300B: arranging each of the at least one spherical marker such that the core center thereof substantially coincides with each of the at least one position to be measured corresponding thereto in the space;

S400B: obtaining and recording relative position data of each of the at least one spherical marker with respect to the designated coordinate system of the vision measuring system and the six-degree position and orientation data of the tracking tool in the same time;

S500B: calculating, based on the relative position data of the spherical marker with respect to the designated coordinate system of the vision measuring system, the calibration relationship between the designated coordinate system of the vision measuring system and the six-degree tracking tool, and the six-degree position and orientation data of the tracking tool, to thereby obtain a 3D space position of the position to be measured for each of the at least one spherical marker in the space.

Herein, the relative position data of the spherical marker with respect to the designated coordinate system of the vision measuring system is substantially the 3D space position of the geometric center (or core center) of the spherical marker with respect to the designated coordinate system of the vision measuring system.

The marker and the tracking assembly can be based on any of the embodiments as described and illustrated above. In one specific application, the dot to be measure is on the skin of a patient, and the marker can comprise a first portion of a CT/MRI signal-strong composition and a second portion of a CT/MRI signal-weak composition, respectively arranged at the core center and elsewhere of the marker.

The marker can also comprise a first portion of a CT/MRI signal poor composition and a second portion of a CT/MRI signal strong composition, respectively arranged at the core center and elsewhere of the marker. With such a way, the image position of the first portion of the marker in a CT/MRI image can be determined easily and accurately with distinguishingly displayed spot. The method thereby can be used to positionally match the 3D position of the marker in physical space with that in CT/MR scan images.

Specifically, with reference to FIGS. 8, 9, and 10, the relative position of the spherical marker 100 (more specifically the geometric center of the spherical marker 100) to be measured is expressed as (x_b, y_b, z_b) with respect to a designated coordinate system in the vision measuring system 213. The coordinate system with its origin in the vision measuring system 213 usually is not just as same as that of the tracking tool. In reference to the coordinate system with its origin of six-degree tracking tool 221, the position of the spherical marker 100 is expressed as (x_s, y_s, z_s). The relationship between (x_b, y_b, z_b)^{T} and (x_s, y_s, z_s)^{T} is:

$$( \begin{matrix}x \_ s \\ y \_ s \\ z \_ s\end{matrix} ) = ( \begin{matrix}Δ x \\ Δ y \\ Δ z\end{matrix} ) + ( \begin{matrix}{r}_{00} & {r}_{01} & {r}_{02} \\ {r}_{10} & {r}_{11} & {r}_{12} \\ {r}_{20} & {r}_{21} & {r}_{22}\end{matrix} ) ( \begin{matrix}x \_ b \\ y \_ b \\ z \_ b\end{matrix} ) \qquad ( 1 )$$

Herein (Δx, Δy, Δz)^{T} represents the translational relationship or offset between the zero point of the designated coordinate system of the vision measuring system 213 and the origin position of the six-degree tracking tool 221, the 3×3 matrix:

$$( \begin{matrix}{r}_{00} & {r}_{01} & {r}_{02} \\ {r}_{10} & {r}_{11} & {r}_{12} \\ {r}_{20} & {r}_{21} & {r}_{22}\end{matrix} )$$

represents the rotational relationship between the designated coordinate system of the vision measuring system 213 and that of the six-degree tracking tool 221.

The six-degree tracking tool 221 is fixedly attached on the vision measuring system 213, then translational and rotational relationship or calibration relationship is constant and can be measured. Through step S200B, (Δx, Δy, Δz)^{T} and the matrix

$$( \begin{matrix}{r}_{00} & {r}_{01} & {r}_{02} \\ {r}_{10} & {r}_{11} & {r}_{12} \\ {r}_{20} & {r}_{21} & {r}_{22}\end{matrix} )$$

of the calibration relationship can be obtained. Through step S400B, the position of (x_b, y_b, z_b) can be obtained.

Further, the six-degree tracking tool 221 is part of a tracking assembly. Regarding the tracking assembly, the spherical marker's position is expressed as (x_t, y_t, z_t) with respect to the reference coordinate system of the tracking assembly. The relationship between (x_t, y_t, z_t)^{T} and (x_s, y_s, z_s)^{T} is:

$$( \begin{matrix}x \_ t \\ y \_ t \\ z \_ t\end{matrix} ) = ( \begin{matrix}{x}^{′} \\ {y}^{′} \\ {z}^{′}\end{matrix} ) + ( \begin{matrix}{m}_{00} & {m}_{01} & {m}_{02} \\ {m}_{10} & {m}_{11} & {m}_{12} \\ {m}_{20} & {m}_{21} & {m}_{22}\end{matrix} ) ( \begin{matrix}x \_ s \\ y \_ s \\ z \_ s\end{matrix} ) \qquad ( 2 )$$

where (x′, y′, z′)^{T} represents the position of the tracking tool 221 with respect to the tracking assembly's coordinate system, where the 3×3 matrix:

$$( \begin{matrix}{m}_{00} & {m}_{01} & {m}_{02} \\ {m}_{10} & {m}_{11} & {m}_{12} \\ {m}_{20} & {m}_{21} & {m}_{22}\end{matrix} )$$

represents the tracking tool 221's direction or rotational matrix regarding to the tracking assembly's coordinate system.

In step S400B, the tracking assembly provides the tracking tool's position (x′, y′, z′), and the direction matrix

$$( \begin{matrix}{m}_{00} & {m}_{01} & {m}_{02} \\ {m}_{10} & {m}_{11} & {m}_{12} \\ {m}_{20} & {m}_{21} & {m}_{22}\end{matrix} ) .$$

By substituting (x_s, y_s, z_s)^{T} in formula (2) with (x_s, y_s, z_s)^{T} in formula (1), the (x_t, y_t, z_t) can be obtained via the follow formula (3):

$$( \begin{matrix}{x}_{t} \\ {y}_{t} \\ {z}_{t}\end{matrix} ) = ( \begin{matrix}{x}^{′} \\ {y}^{′} \\ {z}^{′}\end{matrix} ) + ( \begin{matrix}{m}_{00} & {m}_{01} & {m}_{02} \\ {m}_{10} & {m}_{11} & {m}_{12} \\ {m}_{20} & {m}_{21} & {m}_{22}\end{matrix} ) ( \begin{matrix}Δ x \\ Δ y \\ Δ z\end{matrix} ) + ( \begin{matrix}{m}_{00} & {m}_{01} & {m}_{02} \\ {m}_{10} & {m}_{11} & {m}_{12} \\ {m}_{20} & {m}_{21} & {m}_{22}\end{matrix} ) ( \begin{matrix}{r}_{00} & {r}_{01} & {r}_{02} \\ {r}_{10} & {r}_{11} & {r}_{12} \\ {r}_{20} & {r}_{21} & {r}_{22}\end{matrix} ) ( \begin{matrix}x \_ b \\ y \_ b \\ z \_ b\end{matrix} ) \qquad ( 3 )$$

Formula (3) is just a relationship of the 3D position in the vision measuring system 213 and 3D position in the tracking assembly.

Finally, the position of the core center of the sphere marker (x_t, y_t, z_t) is obtained with respect to the coordinate system of the tracking assembly.

According to some embodiments, the coordinate system of a tracking assembly is based on the transmitter (as illustrated in FIG. 10) as an absolute coordinate system.

The coordinate system of a tracking assembly can also be based on other base reference tracking tool as well. According to some embodiments, the said coordinate system of a tracking assembly is set with a tracking sensor/or tracking tool. This type of tracking sensors or tracking tools are considered as reference tracking sensors or tracking tools. The reference tracking sensor/or tracking tool's position and orientation data is used as a coordinate system for a tracking assembly.

It is noted that in some applications, a larger area, not just one individual position, needs measurement for determining the spatial position and conformation thereof. Without considering its inside structure, the area to be measured can be regarded to include a plurality of positions, configured such that each dot is on a different position on the area, and the plurality of dots together can sufficiently represent the area. By measuring the position of each of the plurality of dots on the area, the spatial position and conformation and of the area can be approximately determined.

For this purpose, this second embodiment of the marker position measuring system as described above can also be utilized for determining a spatial position and conformation of an area to be measure in a space.

It is noted that in some embodiments, those spherical markers can preferably be configured to have special characters depending on the specific position of the corresponding dot on the area to be measured in the space. Among these special characters include geometric, color characters, etc. For example, spherical markers are configured to be on the sharp edges, or with special color characters, such that images of spherical markers can be identified easily from vision measuring system.

It is further noted that in addition to the above embodiment where a plurality of spherical markers are included in the marker tracking assembly, it is possible that the marker position measuring system comprises only one marker, instead of a plurality of markers, and this marker can be repeatedly used to measure a 3D position of each of the set of dots with designated positions on the area, to thereby obtain the spatial position and conformation of an area to be measured in the space.

In some embodiments, an area comprising at least four markers of targets can be measured with respect to a reference coordinate system of the tracking assembly. The area is configured to be rigid, such that each target has a rigid fixed position relatively to each other and the origin and direction of the reference coordinate system of the tracking assembly is arranged at a rigid fixed position and direction relatively to the group positions of the at least four markers of targets; The said reference coordinate system of the tracking assembly can be set with a tracking sensor/or tracking tool. For example, in a case of using of an electromagnetic tracking system, a tracking coil sensor is used as reference base/coordinate system. The said rigid area can also have a room with special position and direction to rigidly place the tracking tool, wherein the room is configured to be rigid fixed, such that the origin and direction of the reference coordinate system of the tracking assembly is rigid fixed whenever the reference tracking tool is placed on or not.

It is known that at least four positions (not coplanar) are needed in order to calculate the transformation of directions and positions between two spaces of two coordinate systems (for example, in physical world and in imaging world). The transformation is a key factor for a surgical navigation system. The positions in imaging world is obtained by scanning the said rigid area and a patient/an object together. The patient could be just the region for operation. During the imaging scanning, the relative position and direction between the patient's operation region and the rigid area are rigid fixed each other. In other words, the patient is rigid fixed relatively to the origin and direction of the reference coordinate system or the room to place the reference tracking tool, and to the groups of the at least four markers of targets. To meet the requirement of rigid position and direction relationship, the rigid area can be placed rigidly on the patient in some way.

By correlating the least four targets' positions in the physical world with that in the imaging world, the transformation is obtained so as to convert any position in the physical world into that in the imaging world. Then during a surgical procedure aided with a navigation system, a medical instrument with its position tracked by a tracking assembly can be displayed in the image with pre-scanned patient images together, providing that the relative position and direction between the patient or the operation region and the origin and direction of the reference coordinate system of the tracking assembly or the room to place the reference tracking tool are rigid fixed and unchanged from the above patient's scanning step, meanwhile the group of the at least four markers are not necessarily presented.

Moreover, it is noted that in any embodiments of the non-contacting method as described above and illustrated in FIG. 11, the calibration relationship between a designated coordinate system of the vision measuring system and the coordinate system of the six-degree tracking tool needs to be determined. There is a translational relationship between the zero point of the designated coordinate system of the vision measuring system and the origin position of the six-degree tracking tool or sensor. There is also a rotational relationship between coordinate axis of the coordinate system of the vision measuring system and the directions of the six-degree sensor or tracking tool.

The present disclosure further provides a method for obtaining a calibration relationship between a designated coordinate system of the vision measuring system and the tracking tool (e.g., step 200B). The method substantially can be used to determine the calibration parameters.

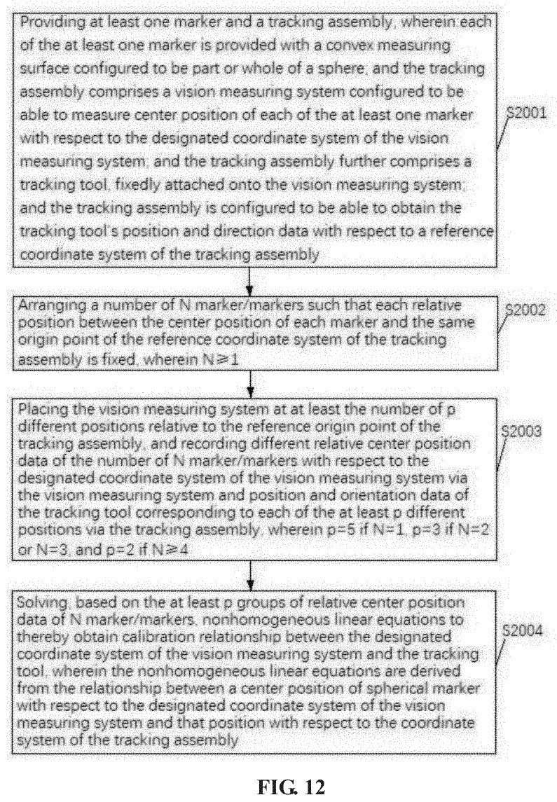

Specifically, according to some embodiments of the disclosure as illustrated in FIG. 12, the method comprises the following steps.

S2001: providing a marker position measuring system comprising at least one marker and a tracking assembly, wherein: each of the at least one marker is provided with a measuring surface configured to be part or whole of a sphere; and the tracking assembly comprises a vision measuring system configured to be able to measure position of a core center of each of the at least one marker with respect to a designated coordinate system of the vision measuring system; and the tracking assembly further comprises a tracking tool, fixedly attached onto the vision measuring system; and the tracking assembly is configured to be able to obtain the tracking tool's position and direction data with respect to a reference coordinate system of the tracking assembly.

S2002: arranging a number of N marker/markers such that each relative position between the position of core center of the each marker and the same origin point of the reference coordinate system of the tracking assembly is fixed, where

The reference point of the tracking assembly can be on the reference tracking sensor or on the transmitter for an electromagnetic tracking assembly. It can also be on the camera assembly for a light/infrared tracking assembly. Herein the designated reference point of the tracking assembly is in the same position for all of the number of marker/markers.

According to some embodiments, the marker is configured to have such a character that, the marker can be easily and uniquely identified and thus has a unique 3D position. For example, on two images by the two cameras of the binocular vision measuring system, the spherical marker pair can be easily and uniquely identified and thus obtain a unique 3D position.

In some embodiments, a marker with a convex surface that is part or whole of a sphere can be used to represent the dot, with the dot at the core center of the sphere.

S2003: placing the vision measuring system, configured to be able to measure 3D positions of N marker/markers at once, at at least the number of p different positions relative to the number of N marker/markers, or relative to the reference origin point of the tracking assembly, and recording, for each placing position of the vision measuring system, different relative position data of core center of the number of N marker/markers with respect to the designated coordinate system of the vision measuring system via the vision measuring system and position and orientation data of the tracking tool corresponding to each of the at least “p” different positions via the tracking assembly, wherein p=5 if N=1, p=3 if N=2 or N=3, and p=2 if N≥4.

Herein the vision measuring system can measure 3D position data of core center of the number of N marker/markers at once and it measures at least p times at at least p different positions relative to the reference origin point of the tracking assembly.

Moreover, when there is more than one marker (N>1), the vision measuring system is configured to be able to identify each individual marker when measure multiple markers by multiple times.

Recall formula (3), the relationship of 3D position in the vision measuring system and in the tracking assembly. On each position's obtaining and recording, there will be known marker/dot position (x_b, y_b, z_b) via the vision measuring system and known tracking tool's data (x′, y′, z′) and matrix:

$$( \begin{matrix}{m}_{00} & {m}_{01} & {m}_{02} \\ {m}_{10} & {m}_{11} & {m}_{12} \\ {m}_{20} & {m}_{21} & {m}_{22}\end{matrix} )$$

via the tracking assemble.

On p^{th} position's getting and recording, there will be known marker/dot position (x^{p}_b, y^{p}_b, z^{p}_b) and known tracking tool's data (xₚ′, yₚ′, zₚ′), and matrix

$$( \begin{matrix}{m}_{00}^{p} & {m}_{01}^{p} & {m}_{02}^{p} \\ {m}_{10}^{p} & {m}_{11}^{p} & {m}_{12}^{p} \\ {m}_{20}^{p} & {m}_{21}^{p} & {m}_{22}^{p}\end{matrix} )$$

In formula (3), there are 12 constant and unknown parameters for (Δx, Δy, Δz) and the matrix

$$( \begin{matrix}{r}_{00} & {r}_{01} & {r}_{02} \\ {r}_{10} & {r}_{11} & {r}_{12} \\ {r}_{20} & {r}_{21} & {r}_{22}\end{matrix} ) .$$

As mentioned before the vision measuring system measures 3D position data of core center of the number of N marker/markers at once on each position placing of the vision measuring system. For the number of N marker/markers, there will be known marker position (x_b_{N}, y_b_{N}, z_b_{N}) for N^{th} marker/markers and the same known tracking tool's data (x′, y′, z′) and matrix

$$( \begin{matrix}{m}_{00} & {m}_{01} & {m}_{02} \\ {m}_{10} & {m}_{11} & {m}_{12} \\ {m}_{20} & {m}_{21} & {m}_{22}\end{matrix} )$$

for each position placing of the vision measuring system. There are N×3 constants and unknown data of (x^{N}_t, y^{N}_t, z^{N}_t), and 12 constant and unknown parameters for (Δx, Δy, Δz) and

$$( \begin{matrix}{r}_{00} & {r}_{01} & {r}_{02} \\ {r}_{10} & {r}_{11} & {r}_{12} \\ {r}_{20} & {r}_{21} & {r}_{22}\end{matrix} ) ,$$

wherein (x^{N}_t, y^{N}_t, z^{N}_t) represents the N^{th} marker's 3D position with regard to the reference origin point of the tracking assembly.

Thereby there are number of N equation group of formula (3), or the number of Nx3 equations for each position measuring of the vision measuring system:

$$( \begin{matrix}{x}_{\_ t}^{1} \\ {y}_{\_ t}^{1} \\ {z}_{\_ t}^{1}\end{matrix} ) = ( \begin{matrix}{x}^{′} \\ {y}^{′} \\ {z}^{′}\end{matrix} ) + ( \begin{matrix}{m}_{00} & {m}_{01} & {m}_{02} \\ {m}_{10} & {m}_{11} & {m}_{12} \\ {m}_{20} & {m}_{21} & {m}_{22}\end{matrix} ) ( \begin{matrix}Δ x \\ Δ y \\ Δ z\end{matrix} ) + ( \begin{matrix}{m}_{00} & {m}_{01} & {m}_{02} \\ {m}_{10} & {m}_{11} & {m}_{12} \\ {m}_{20} & {m}_{21} & {m}_{22}\end{matrix} ) ( \begin{matrix}{r}_{00} & {r}_{01} & {r}_{02} \\ {r}_{10} & {r}_{11} & {r}_{12} \\ {r}_{20} & {r}_{21} & {r}_{22}\end{matrix} ) ( \begin{matrix}{x}_{\_ b 1} \\ {y}_{\_ b 1} \\ {z}_{\_ b 1}\end{matrix} ) \\ ( \begin{matrix}{x}_{\_ t}^{2} \\ {y}_{\_ t}^{2} \\ {z}_{\_ t}^{2}\end{matrix} ) = ( \begin{matrix}{x}^{′} \\ {y}^{′} \\ {z}^{′}\end{matrix} ) + ( \begin{matrix}{m}_{00} & {m}_{01} & {m}_{02} \\ {m}_{10} & {m}_{11} & {m}_{12} \\ {m}_{20} & {m}_{21} & {m}_{22}\end{matrix} ) ( \begin{matrix}Δ x \\ Δ y \\ Δ z\end{matrix} ) + ( \begin{matrix}{m}_{00} & {m}_{01} & {m}_{02} \\ {m}_{10} & {m}_{11} & {m}_{12} \\ {m}_{20} & {m}_{21} & {m}_{22}\end{matrix} ) ( \begin{matrix}{r}_{00} & {r}_{01} & {r}_{02} \\ {r}_{10} & {r}_{11} & {r}_{12} \\ {r}_{20} & {r}_{21} & {r}_{22}\end{matrix} ) ( \begin{matrix}{x}_{\_ b 2} \\ {y}_{\_ b 2} \\ {z}_{\_ b 2}\end{matrix} ) \\ ………… \\ ( \begin{matrix}{x}_{\_ t}^{N} \\ {y}_{\_ t}^{N} \\ {z}_{\_ t}^{N}\end{matrix} ) = ( \begin{matrix}{x}^{′} \\ {y}^{′} \\ {z}^{′}\end{matrix} ) + ( \begin{matrix}{m}_{00} & {m}_{01} & {m}_{02} \\ {m}_{10} & {m}_{11} & {m}_{12} \\ {m}_{20} & {m}_{21} & {m}_{22}\end{matrix} ) ( \begin{matrix}Δ x \\ Δ y \\ Δ z\end{matrix} ) + ( \begin{matrix}{m}_{00} & {m}_{01} & {m}_{02} \\ {m}_{10} & {m}_{11} & {m}_{12} \\ {m}_{20} & {m}_{21} & {m}_{22}\end{matrix} ) ( \begin{matrix}{r}_{00} & {r}_{01} & {r}_{02} \\ {r}_{10} & {r}_{11} & {r}_{12} \\ {r}_{20} & {r}_{21} & {r}_{22}\end{matrix} ) ( \begin{matrix}{x}_{\_ bN} \\ {y}_{\_ bN} \\ {z}_{\_ bN}\end{matrix} ) \qquad ( 4 )$$

where N≥1.

Since the vision measuring system measures at least p times at at least p different positions relative to the reference origin point of the tracking assembly, there are at least p×N equation group of formula (3), or at least p×N×3 equations:

$$( \begin{matrix}{x}_{\_ t}^{1} \\ {y}_{\_ t}^{1} \\ {z}_{\_ t}^{1}\end{matrix} ) = ( \begin{matrix}{x}_{1}^{′} \\ {y}_{1}^{′} \\ {z}_{1}^{′}\end{matrix} ) + ( \begin{matrix}{m}_{00}^{1} & {m}_{01}^{1} & {m}_{02}^{1} \\ {m}_{10}^{1} & {m}_{11}^{1} & {m}_{12}^{1} \\ {m}_{20}^{1} & {m}_{21}^{1} & {m}_{22}^{1}\end{matrix} ) ( \begin{matrix}Δ x \\ Δ y \\ Δ z\end{matrix} ) + ( \begin{matrix}{m}_{00}^{1} & {m}_{01}^{1} & {m}_{02}^{1} \\ {m}_{10}^{1} & {m}_{11}^{1} & {m}_{12}^{1} \\ {m}_{20}^{1} & {m}_{21}^{1} & {m}_{22}^{1}\end{matrix} ) ( \begin{matrix}{r}_{00} & {r}_{01} & {r}_{02} \\ {r}_{10} & {r}_{11} & {r}_{12} \\ {r}_{20} & {r}_{21} & {r}_{22}\end{matrix} ) ( \begin{matrix}{x}_{\_ b 1}^{1} \\ {y}_{\_ b 1}^{1} \\ {z}_{\_ b 1}^{1}\end{matrix} ) \\ ( \begin{matrix}{x}_{\_ t}^{2} \\ {y}_{\_ t}^{2} \\ {z}_{\_ t}^{2}\end{matrix} ) = ( \begin{matrix}{x}_{1}^{′} \\ {y}_{1}^{′} \\ {z}_{1}^{′}\end{matrix} ) + ( \begin{matrix}{m}_{00}^{1} & {m}_{01}^{1} & {m}_{02}^{1} \\ {m}_{10}^{1} & {m}_{11}^{1} & {m}_{12}^{1} \\ {m}_{20}^{1} & {m}_{21}^{1} & {m}_{22}^{1}\end{matrix} ) ( \begin{matrix}Δ x \\ Δ y \\ Δ z\end{matrix} ) + ( \begin{matrix}{m}_{00}^{1} & {m}_{01}^{1} & {m}_{02}^{1} \\ {m}_{10}^{1} & {m}_{11}^{1} & {m}_{12}^{1} \\ {m}_{20}^{1} & {m}_{21}^{1} & {m}_{22}^{1}\end{matrix} ) ( \begin{matrix}{r}_{00} & {r}_{01} & {r}_{02} \\ {r}_{10} & {r}_{11} & {r}_{12} \\ {r}_{20} & {r}_{21} & {r}_{22}\end{matrix} ) ( \begin{matrix}{x}_{\_ b 2}^{1} \\ {y}_{\_ b 2}^{1} \\ {z}_{\_ b 2}^{1}\end{matrix} ) \\ ………… \\ ( \begin{matrix}{x}_{\_ t}^{N} \\ {y}_{\_ t}^{N} \\ {z}_{\_ t}^{N}\end{matrix} ) = ( \begin{matrix}{x}_{1}^{′} \\ {y}_{1}^{′} \\ {z}_{1}^{′}\end{matrix} ) + ( \begin{matrix}{m}_{00}^{1} & {m}_{01}^{1} & {m}_{02}^{1} \\ {m}_{10}^{1} & {m}_{11}^{1} & {m}_{12}^{1} \\ {m}_{20}^{1} & {m}_{21}^{1} & {m}_{22}^{1}\end{matrix} ) ( \begin{matrix}Δ x \\ Δ y \\ Δ z\end{matrix} ) + ( \begin{matrix}{m}_{00}^{1} & {m}_{01}^{1} & {m}_{02}^{1} \\ {m}_{10}^{1} & {m}_{11}^{1} & {m}_{12}^{1} \\ {m}_{20}^{1} & {m}_{21}^{1} & {m}_{22}^{1}\end{matrix} ) ( \begin{matrix}{r}_{00} & {r}_{01} & {r}_{02} \\ {r}_{10} & {r}_{11} & {r}_{12} \\ {r}_{20} & {r}_{21} & {r}_{22}\end{matrix} ) ( \begin{matrix}{x}_{\_ bN}^{1} \\ {y}_{\_ bN}^{1} \\ {z}_{\_ bN}^{1}\end{matrix} ) \\ ( \begin{matrix}{x}_{\_ t}^{1} \\ {y}_{\_ t}^{1} \\ {z}_{\_ t}^{1}\end{matrix} ) = ( \begin{matrix}{x}_{2}^{′} \\ {y}_{2}^{′} \\ {z}_{2}^{′}\end{matrix} ) + ( \begin{matrix}{m}_{00}^{2} & {m}_{01}^{2} & {m}_{02}^{2} \\ {m}_{10}^{2} & {m}_{11}^{2} & {m}_{12}^{2} \\ {m}_{20}^{2} & {m}_{21}^{2} & {m}_{22}^{2}\end{matrix} ) ( \begin{matrix}Δ x \\ Δ y \\ Δ z\end{matrix} ) + ( \begin{matrix}{m}_{00}^{2} & {m}_{01}^{2} & {m}_{02}^{2} \\ {m}_{10}^{2} & {m}_{11}^{2} & {m}_{12}^{2} \\ {m}_{20}^{2} & {m}_{21}^{2} & {m}_{22}^{2}\end{matrix} ) ( \begin{matrix}{r}_{00} & {r}_{01} & {r}_{02} \\ {r}_{10} & {r}_{11} & {r}_{12} \\ {r}_{20} & {r}_{21} & {r}_{22}\end{matrix} ) ( \begin{matrix}{x}_{\_ b 1}^{2} \\ {y}_{\_ b 1}^{2} \\ {z}_{\_ b 1}^{2}\end{matrix} ) \\ ( \begin{matrix}{x}_{\_ t}^{2} \\ {y}_{\_ t}^{2} \\ {z}_{\_ t}^{2}\end{matrix} ) = ( \begin{matrix}{x}_{2}^{′} \\ {y}_{2}^{′} \\ {z}_{2}^{′}\end{matrix} ) + ( \begin{matrix}{m}_{00}^{2} & {m}_{01}^{2} & {m}_{02}^{2} \\ {m}_{10}^{2} & {m}_{11}^{2} & {m}_{12}^{2} \\ {m}_{20}^{2} & {m}_{21}^{2} & {m}_{22}^{2}\end{matrix} ) ( \begin{matrix}Δ x \\ Δ y \\ Δ z\end{matrix} ) + ( \begin{matrix}{m}_{00}^{2} & {m}_{01}^{2} & {m}_{02}^{2} \\ {m}_{10}^{2} & {m}_{11}^{2} & {m}_{12}^{2} \\ {m}_{20}^{2} & {m}_{21}^{2} & {m}_{22}^{2}\end{matrix} ) ( \begin{matrix}{r}_{00} & {r}_{01} & {r}_{02} \\ {r}_{10} & {r}_{11} & {r}_{12} \\ {r}_{20} & {r}_{21} & {r}_{22}\end{matrix} ) ( \begin{matrix}{x}_{\_ b 2}^{2} \\ {y}_{\_ b 2}^{2} \\ {z}_{\_ b 2}^{2}\end{matrix} ) \\ ………… \\ ( \begin{matrix}{x}_{\_ t}^{N} \\ {y}_{\_ t}^{N} \\ {z}_{\_ t}^{N}\end{matrix} ) = ( \begin{matrix}{x}_{2}^{′} \\ {y}_{2}^{′} \\ {z}_{2}^{′}\end{matrix} ) + ( \begin{matrix}{m}_{00}^{2} & {m}_{01}^{2} & {m}_{02}^{2} \\ {m}_{10}^{2} & {m}_{11}^{2} & {m}_{12}^{2} \\ {m}_{20}^{2} & {m}_{21}^{2} & {m}_{22}^{2}\end{matrix} ) ( \begin{matrix}Δ x \\ Δ y \\ Δ z\end{matrix} ) + ( \begin{matrix}{m}_{00}^{2} & {m}_{01}^{2} & {m}_{02}^{2} \\ {m}_{10}^{2} & {m}_{11}^{2} & {m}_{12}^{2} \\ {m}_{20}^{2} & {m}_{21}^{2} & {m}_{22}^{2}\end{matrix} ) ( \begin{matrix}{r}_{00} & {r}_{01} & {r}_{02} \\ {r}_{10} & {r}_{11} & {r}_{12} \\ {r}_{20} & {r}_{21} & {r}_{22}\end{matrix} ) ( \begin{matrix}{x}_{\_ bN}^{2} \\ {y}_{\_ bN}^{2} \\ {z}_{\_ bN}^{2}\end{matrix} ) \\ ………… \\ ( \begin{matrix}{x}_{\_ t}^{1} \\ {y}_{\_ t}^{1} \\ {z}_{\_ t}^{1}\end{matrix} ) = ( \begin{matrix}{x}_{p}^{′} \\ {y}_{p}^{′} \\ {z}_{p}^{′}\end{matrix} ) + ( \begin{matrix}{m}_{00}^{p} & {m}_{01}^{p} & {m}_{02}^{p} \\ {m}_{10}^{p} & {m}_{11}^{p} & {m}_{12}^{p} \\ {m}_{20}^{p} & {m}_{21}^{p} & {m}_{22}^{p}\end{matrix} ) ( \begin{matrix}Δ x \\ Δ y \\ Δ z\end{matrix} ) + ( \begin{matrix}{m}_{00}^{p} & {m}_{01}^{p} & {m}_{02}^{p} \\ {m}_{10}^{p} & {m}_{11}^{p} & {m}_{12}^{p} \\ {m}_{20}^{p} & {m}_{21}^{p} & {m}_{22}^{p}\end{matrix} ) ( \begin{matrix}{r}_{00} & {r}_{01} & {r}_{02} \\ {r}_{10} & {r}_{11} & {r}_{12} \\ {r}_{20} & {r}_{21} & {r}_{22}\end{matrix} ) ( \begin{matrix}{x}_{\_ b 1}^{p} \\ {y}_{\_ b 1}^{p} \\ {z}_{\_ b 1}^{p}\end{matrix} ) \\ ( \begin{matrix}{x}_{\_ t}^{2} \\ {y}_{\_ t}^{2} \\ {z}_{\_ t}^{2}\end{matrix} ) = ( \begin{matrix}{x}_{p}^{′} \\ {y}_{p}^{′} \\ {z}_{p}^{′}\end{matrix} ) + ( \begin{matrix}{m}_{00}^{p} & {m}_{01}^{p} & {m}_{02}^{p} \\ {m}_{10}^{p} & {m}_{11}^{p} & {m}_{12}^{p} \\ {m}_{20}^{p} & {m}_{21}^{p} & {m}_{22}^{p}\end{matrix} ) ( \begin{matrix}Δ x \\ Δ y \\ Δ z\end{matrix} ) + ( \begin{matrix}{m}_{00}^{p} & {m}_{01}^{p} & {m}_{02}^{p} \\ {m}_{10}^{p} & {m}_{11}^{p} & {m}_{12}^{p} \\ {m}_{20}^{p} & {m}_{21}^{p} & {m}_{22}^{p}\end{matrix} ) ( \begin{matrix}{r}_{00} & {r}_{01} & {r}_{02} \\ {r}_{10} & {r}_{11} & {r}_{12} \\ {r}_{20} & {r}_{21} & {r}_{22}\end{matrix} ) ( \begin{matrix}{x}_{\_ b 2}^{p} \\ {y}_{\_ b 2}^{p} \\ {z}_{\_ b 2}^{p}\end{matrix} ) \\ ………… \\ ( \begin{matrix}{x}_{\_ t}^{N} \\ {y}_{\_ t}^{N} \\ {z}_{\_ t}^{N}\end{matrix} ) = ( \begin{matrix}{x}_{p}^{′} \\ {y}_{p}^{′} \\ {z}_{p}^{′}\end{matrix} ) + ( \begin{matrix}{m}_{00}^{p} & {m}_{01}^{p} & {m}_{02}^{p} \\ {m}_{10}^{p} & {m}_{11}^{p} & {m}_{12}^{p} \\ {m}_{20}^{p} & {m}_{21}^{p} & {m}_{22}^{p}\end{matrix} ) ( \begin{matrix}Δ x \\ Δ y \\ Δ z\end{matrix} ) + ( \begin{matrix}{m}_{00}^{p} & {m}_{01}^{p} & {m}_{02}^{p} \\ {m}_{10}^{p} & {m}_{11}^{p} & {m}_{12}^{p} \\ {m}_{20}^{p} & {m}_{21}^{p} & {m}_{22}^{p}\end{matrix} ) ( \begin{matrix}{r}_{00} & {r}_{01} & {r}_{02} \\ {r}_{10} & {r}_{11} & {r}_{12} \\ {r}_{20} & {r}_{21} & {r}_{22}\end{matrix} ) ( \begin{matrix}{x}_{\_ bN}^{p} \\ {y}_{\_ bN}^{p} \\ {z}_{\_ bN}^{p}\end{matrix} ) \qquad ( 5 )$$

wherein p represents p^{th} time to get and record data at different p times position, p=5 if N=1, p=3 if N=2 or N=3, and p=2 if N≥4.

S2004: solving, based on the at least p groups of data of N marker/markers obtained in S2003, the number of p×N×3 nonhomogeneous linear equations to thereby obtain calibration relationship between the designated coordinate system of the vision measuring system and the tracking tool, wherein the nonhomogeneous linear equations are derived from the relationship between the 3D position in the vision measuring system and the 3D position in the tracking assembly.

From the relationship between 3D position in the vision measuring system and 3D position in the tracking assemble, herein expressed with formula (3), the nonhomogeneous linear equations, i.e. formula (5) can be derived, wherein there are p×N×3 equations with N×3+12 constant and unknown parameters for the number of N 3D positions of (x^{N}_t, y^{N}_t, z^{N}A, (Δx, Δy, Δz) and the matrix

$$( \begin{matrix}{r}_{00} & {r}_{01} & {r}_{02} \\ {r}_{10} & {r}_{11} & {r}_{12} \\ {r}_{20} & {r}_{21} & {r}_{22}\end{matrix} ) ,$$

wherein (x^{N}_t, y^{N}_t, z^{N}_t) represents the marker N^{th} 3D position with regard to the reference origin point of the tracking assembly. It is noted that the condition of p=5 if N=1, p=3 if N=2 or N=3, and p=2 if N≥4 makes the equation number of p×N×3 is always larger than the constant and unknown parameters' number of N×3+12. By solving nonhomogeneous linear equations (5), two results are obtained, including:

a position offset: (Δx, Δy, Δz), i.e., the translational relationship between the origin point of the designated coordinate system of the vision measuring system and the origin position of the six-degree tracking tool; and

a matrix:

$$( \begin{matrix}{r}_{00} & {r}_{01} & {r}_{02} \\ {r}_{10} & {r}_{11} & {r}_{12} \\ {r}_{20} & {r}_{21} & {r}_{22}\end{matrix} )$$

i.e., the rotational relationship between coordinate axis of the coordinate system of the vision measuring system and the directions of the six-degree tracking tool.

At least some embodiments of the system and method for measuring markers can include one or more of the following embodiments.

First, because a core center of a sphere is used to represent a dot, no matter how big the marker is, an improved accuracy can be achieved since only the position data for the core center of the sphere is calculated.

Second, the marker measurement becomes more convenient. The measuring piece's head can be placed on different positions of the measuring surface of the marker which is part or whole of a sphere, and still gives same position data, which is substantially position data of the core center of the sphere.

Third, the system can be used to realize a non-contacting measurement which does not cause any movement of the object to be measured. The vision measuring system can view the spherical marker(s) from different directions and different distance in a non-contact manner, while it still gives a substantially same position data, which is the position data of the core center of the sphere.

Fourth, measuring a bigger area becomes available and easy. The relative position between the measured object and the measuring system is flexible. The measurement is with a free hand way. No matter where the vision measuring system is, the tracking assembly provides the unique measuring base.

The embodiments disclosed here may be applicable to cases that need measurements of a position, a dot-liked object, or surface of an object with contacting pen or non-contacting vision system with a tracking assembly.

The tracking system can employ one or more different types of positioning methods and devices, such optical devices that employ light or infrared (IR) beams such as laser beams for positioning, an active or passive tracking system, magnetic tracking, radio frequency (RF) tracking, ultrasound tracking, etc.

Those of ordinary skill in the art will recognize that the functional blocks, methods, units, devices, and systems described in the present disclosure may be integrated or divided into different combinations of systems, units, devices, and functional blocks. Any suitable programming languages and programming techniques may be used to implement the routines of particular embodiments. Different programming techniques may be employed such as procedural or object-oriented. The routines may execute on a single processing device or multiple processors. Although the steps, operations, or computations may be presented in a specific order, the order may be changed in different particular embodiments. In some particular embodiments, multiple steps shown as sequential in this disclosure may be performed at the same time.

The “processor” or “processing circuit” can include any suitable hardware and/or software system, mechanism or component that processes data, signals or other information. The processor may include a system with a general-purpose central processing unit, multiple processing units, dedicated circuitry for achieving functionality, or other systems. Processing needs not be limited to a geographic location, or have temporal limitations. For example, a processor may perform its functions in “real-time,” “offline,” in a “batch mode,” etc. Portions of processing may be performed at different times and at different locations, by different (or the same) processing systems. Various embodiments disclosed herein can be realized via hardware and/or software, such as a computer program stored on a memory. For example, a tangible, non-transitory, computer-readable storage medium having instructions stored there on that, when executed by one or more processors, cause the one or more processors to perform operations including the steps described above.

In some embodiments, a software or program code is provided to realize the method described above. The software or program code can be stored on any type of computer-readable medium or memory, such as a storage device including a disk or hard drive. The computer-readable medium may include a non-transitory computer-readable medium or memory, such as computer-readable media that store data for short periods of time like register memory, processor cache and Random-Access Memory (RAM). The computer-readable medium may also include non-transitory media or memory, such as secondary or persistent long-term storage, like read only memory (ROM), optical or magnetic disks, compact-disc read only memory (CD-ROM), for example. The computer readable media may also be any other volatile or non-volatile storage systems. The computer readable medium may be considered a computer readable storage medium, a tangible storage device, or other article of manufacture, for example. The software instructions can be stored in the computer readable media, and also be contained in, and provided as, an electronic signal, for example in the form of software as a service (SaaS) delivered from a server (e.g., a distributed system and/or a cloud computing system).

Although specific embodiments have been described above in detail, the description is merely for purposes of illustration. It should be appreciated, therefore, that many aspects described above are not intended as required or essential elements unless explicitly stated otherwise.

Various modifications of, and equivalent acts corresponding to, the disclosed aspects of the exemplary embodiments, in addition to those described above, can be made by a person of ordinary skill in the art, having the benefit of the present disclosure, without departing from the spirit and scope of the disclosure defined in the following claims, the scope of which is to be accorded the broadest interpretation so as to encompass such modifications and equivalent structures.

1. A method of measuring at least one target's position, the method comprising:
a) providing a marker for each target and a tracking assembly, wherein:
each marker has a convex measuring surface, configured to be part or whole of a sphere, such that the center of the convex measuring surface substantially corresponds to the position of the target to be measured; and
the tracking assembly comprises a measuring piece; and
the tracking assembly further comprises a tracking tool, fixedly attached onto the measuring piece; and
the measuring piece is configured to be able to obtain the center position of the convex measuring surface of the marker/markers with respect to the tracking tool; and
the tracking assembly is configured to be able to obtain the tracking tool's position and direction data with respect to a reference coordinate system of the tracking assembly;
b) obtaining and recording the center position data of the convex measuring surface of each of the at least one marker with respect to the tracking tool and the tracking tool's position and orientation data with respect to the reference coordinate system of the tracking assembly;
c) calculating, based on the recorded center position data of the convex measuring surface of each of the at least one marker with respect to the tracking tool, and the recorded tracking tool's position and orientation data with respect to the reference coordinate system of the tracking assembly, to thereby obtain each target's the position with respect to the reference coordinate system of the tracking assembly.

2. The method of claim 1, wherein the measuring piece has a concave measuring surface substantially fit with the convex measuring surface of each of the at least one marker; and the measuring piece is configured to be able to obtain the center position of the concave surface with respect to the tracking tool.

3. The method of claim 2, wherein the obtaining and recording the center position data of the convex measuring surface of each of the at least one marker with respect to the tracking tool and the tracking tool's position and orientation data with respect to the reference coordinate system of the tracking assembly is by contacting the concave surface of the measuring piece with the convex measuring surface of each of the at least one marker.

4. The method of claim 1, wherein the measuring piece comprises a vision measuring system configured to be able to measure position of a center of each of the at least one marker with respect to a designated coordinate system of the vision measuring system; and
the calibration relationship between the designated coordinate system of the vision measuring system and the tracking tool is known;

5. The method of claim 4, wherein the obtaining and recording the center position data of the convex measuring surface of each of the at least one marker with respect to the tracking tool is based on the measured position of a center of each of the at least one marker with respect to the designated coordinate system of the vision measuring system and the calibration relationship between the designated coordinate system of the vision measuring system and the tracking tool.

6. The method of claim 5, wherein the position of a center of each of the at least one marker with respect to the designated coordinate system of the vision measuring system is expressed as (x_b, y_b, z_b), satisfying a relationship:
$$( \begin{matrix}x \_ s \\ y \_ s \\ z \_ s\end{matrix} ) = ( \begin{matrix}Δ x \\ Δ y \\ Δ z\end{matrix} ) + ( \begin{matrix}{r}_{00} & {r}_{01} & {r}_{02} \\ {r}_{10} & {r}_{11} & {r}_{12} \\ {r}_{20} & {r}_{21} & {r}_{22}\end{matrix} ) ( \begin{matrix}x \_ b \\ y \_ b \\ z \_ b\end{matrix} ) \qquad ( 1 )$$
wherein:
the (Δx, Δy, Δz)^{T} represents an offset between a zero point of the designated coordinate system of the vision measuring system and the position of the tracking tool;
the 3×3 matrix:
$$( \begin{matrix}{r}_{00} & {r}_{01} & {r}_{02} \\ {r}_{10} & {r}_{11} & {r}_{12} \\ {r}_{20} & {r}_{21} & {r}_{22}\end{matrix} )$$
represents a rotational relationship between the designated coordinate system of the vision measuring system and the tracking tool; and
the (x_s, y_s, z_s) represents the center position of each of the at least one marker with respect to the tracking tool.

7. The method of claim 6, wherein the center position of each of the at least one marker with respect to the tracking tool expressed as (x_s, y_s, z_s) is further satisfied with a relationship:
$$( \begin{matrix}x \_ t \\ y \_ t \\ z \_ t\end{matrix} ) = ( \begin{matrix}{x}^{′} \\ {y}^{′} \\ {z}^{′}\end{matrix} ) + ( \begin{matrix}{m}_{00} & {m}_{01} & {m}_{02} \\ {m}_{10} & {m}_{11} & {m}_{12} \\ {m}_{20} & {m}_{21} & {m}_{22}\end{matrix} ) ( \begin{matrix}x \_ s \\ y \_ s \\ z \_ s\end{matrix} ) \qquad ( 2 )$$
wherein:
the (x′, y′, z′)^{T} represents a position of the tracking tool with regard to the tracking assembly's coordinate system; and
the 3×3 matrix:
$$( \begin{matrix}{m}_{00} & {m}_{01} & {m}_{02} \\ {m}_{10} & {m}_{11} & {m}_{12} \\ {m}_{20} & {m}_{21} & {m}_{22}\end{matrix} )$$
represents a rotational relationship between tracking tool and the tracking assembly's coordinate system; and
the (x_t, y_t, z_t) represents a center position of each of the at least one marker with respect to the tracking assembly's coordinate system.

8. The method of claim 7, wherein the calculating, based on the recorded center position data of the convex measuring surface of each of the at least one marker with respect to the tracking tool, and the recorded tracking tool's position and orientation data with respect to the reference coordinate system of the tracking assembly, to thereby obtain each target's the position with respect to the reference coordinate system of the tracking assembly comprises:
substituting (x_s, y_s, z_s)^{T} in formula (2) with (x_s, y_s, z_s)^{T} in formula (1) to obtain a
$$( \begin{matrix}{x}_{t} \\ {y}_{t} \\ {z}_{t}\end{matrix} ) = ( \begin{matrix}{x}^{′} \\ {y}^{′} \\ {z}^{′}\end{matrix} ) + ( \begin{matrix}{m}_{00} & {m}_{01} & {m}_{02} \\ {m}_{10} & {m}_{11} & {m}_{12} \\ {m}_{20} & {m}_{21} & {m}_{22}\end{matrix} ) ( \begin{matrix}Δ x \\ Δ y \\ Δ z\end{matrix} ) + ( \begin{matrix}{m}_{00} & {m}_{01} & {m}_{02} \\ {m}_{10} & {m}_{11} & {m}_{12} \\ {m}_{20} & {m}_{21} & {m}_{22}\end{matrix} ) ( \begin{matrix}{r}_{00} & {r}_{01} & {r}_{02} \\ {r}_{10} & {r}_{11} & {r}_{12} \\ {r}_{20} & {r}_{21} & {r}_{22}\end{matrix} ) ( \begin{matrix}x \_ b \\ y \_ b \\ z \_ b\end{matrix} ) \qquad ( 3 )$$
to thereby calculate the position (x_t, y_t, z_t) of core center of each of the at least one maker in the space with respect to the reference coordinate system of the tracking assembly.

9. The method of claim 4, wherein the calibration relationship between the designated coordinate system of the vision measuring system and the tracking tool is known by a method, the method to determine the calibration relationship comprising
a) providing at least one marker and a tracking assembly, wherein:
each of the at least one marker is provided with a convex measuring surface configured to be part or whole of a sphere; and
the tracking assembly comprises a vision measuring system configured to be able to measure center position of each of the at least one marker with respect to the designated coordinate system of the vision measuring system; and
the tracking assembly further comprises a tracking tool, fixedly attached onto the vision measuring system; and
the tracking assembly is configured to be able to obtain the tracking tool's position and direction data with respect to a reference coordinate system of the tracking assembly;
b) arranging a number of N marker/markers such that each relative position between the center position of each marker and the same origin point of the reference coordinate system of the tracking assembly is fixed, wherein N≥1;
c) placing the vision measuring system at at least the number of p different positions relative to the reference origin point of the tracking assembly, and recording different relative center position data of the number of N marker/markers with respect to the designated coordinate system of the vision measuring system via the vision measuring system and position and orientation data of the tracking tool corresponding to each of the at least p different positions via the tracking assembly, wherein p=5 if N=1, p=3 if N=2 or N=3, and p=2 if N≥4; and
d) solving, based on the at least p groups of relative center position data of N marker/markers, nonhomogeneous linear equations to thereby obtain calibration relationship between the designated coordinate system of the vision measuring system and the tracking tool, wherein the nonhomogeneous linear equations are derived from the relationship between a center position of spherical marker with respect to the designated coordinate system of the vision measuring system and that position with respect to the coordinate system of the tracking assembly.

10. The method of claim 9, wherein the placing the vision measuring system at at least the number of p different positions relative to the reference origin point of the tracking assembly, and recording different relative center position data of the number of N marker/markers with respect to the designated coordinate system of the vision measuring system via the vision measuring system and position and orientation data of the tracking tool corresponding to each of the at least p different positions via the tracking assembly comprises:
obtaining at least p×3×N equations in at least p×N equation groups:
$$( \begin{matrix}{x}_{\_ t}^{1} \\ {y}_{\_ t}^{1} \\ {z}_{\_ t}^{1}\end{matrix} ) = ( \begin{matrix}{x}_{1}^{′} \\ {y}_{1}^{′} \\ {z}_{1}^{′}\end{matrix} ) + ( \begin{matrix}{m}_{00}^{1} & {m}_{01}^{1} & {m}_{02}^{1} \\ {m}_{10}^{1} & {m}_{11}^{1} & {m}_{12}^{1} \\ {m}_{20}^{1} & {m}_{21}^{1} & {m}_{22}^{1}\end{matrix} ) ( \begin{matrix}Δ x \\ Δ y \\ Δ z\end{matrix} ) + ( \begin{matrix}{m}_{00}^{1} & {m}_{01}^{1} & {m}_{02}^{1} \\ {m}_{10}^{1} & {m}_{11}^{1} & {m}_{12}^{1} \\ {m}_{20}^{1} & {m}_{21}^{1} & {m}_{22}^{1}\end{matrix} ) ( \begin{matrix}{r}_{00} & {r}_{01} & {r}_{02} \\ {r}_{10} & {r}_{11} & {r}_{12} \\ {r}_{20} & {r}_{21} & {r}_{22}\end{matrix} ) ( \begin{matrix}{x}_{\_ b 1}^{1} \\ {y}_{\_ b 1}^{1} \\ {z}_{\_ b 1}^{1}\end{matrix} ) \\ ( \begin{matrix}{x}_{\_ t}^{2} \\ {y}_{\_ t}^{2} \\ {z}_{\_ t}^{2}\end{matrix} ) = ( \begin{matrix}{x}_{1}^{′} \\ {y}_{1}^{′} \\ {z}_{1}^{′}\end{matrix} ) + ( \begin{matrix}{m}_{00}^{1} & {m}_{01}^{1} & {m}_{02}^{1} \\ {m}_{10}^{1} & {m}_{11}^{1} & {m}_{12}^{1} \\ {m}_{20}^{1} & {m}_{21}^{1} & {m}_{22}^{1}\end{matrix} ) ( \begin{matrix}Δ x \\ Δ y \\ Δ z\end{matrix} ) + ( \begin{matrix}{m}_{00}^{1} & {m}_{01}^{1} & {m}_{02}^{1} \\ {m}_{10}^{1} & {m}_{11}^{1} & {m}_{12}^{1} \\ {m}_{20}^{1} & {m}_{21}^{1} & {m}_{22}^{1}\end{matrix} ) ( \begin{matrix}{r}_{00} & {r}_{01} & {r}_{02} \\ {r}_{10} & {r}_{11} & {r}_{12} \\ {r}_{20} & {r}_{21} & {r}_{22}\end{matrix} ) ( \begin{matrix}{x}_{\_ b 2}^{1} \\ {y}_{\_ b 2}^{1} \\ {z}_{\_ b 2}^{1}\end{matrix} ) \\ ………… \\ ( \begin{matrix}{x}_{\_ t}^{N} \\ {y}_{\_ t}^{N} \\ {z}_{\_ t}^{N}\end{matrix} ) = ( \begin{matrix}{x}_{1}^{′} \\ {y}_{1}^{′} \\ {z}_{1}^{′}\end{matrix} ) + ( \begin{matrix}{m}_{00}^{1} & {m}_{01}^{1} & {m}_{02}^{1} \\ {m}_{10}^{1} & {m}_{11}^{1} & {m}_{12}^{1} \\ {m}_{20}^{1} & {m}_{21}^{1} & {m}_{22}^{1}\end{matrix} ) ( \begin{matrix}Δ x \\ Δ y \\ Δ z\end{matrix} ) + ( \begin{matrix}{m}_{00}^{1} & {m}_{01}^{1} & {m}_{02}^{1} \\ {m}_{10}^{1} & {m}_{11}^{1} & {m}_{12}^{1} \\ {m}_{20}^{1} & {m}_{21}^{1} & {m}_{22}^{1}\end{matrix} ) ( \begin{matrix}{r}_{00} & {r}_{01} & {r}_{02} \\ {r}_{10} & {r}_{11} & {r}_{12} \\ {r}_{20} & {r}_{21} & {r}_{22}\end{matrix} ) ( \begin{matrix}{x}_{\_ bN}^{1} \\ {y}_{\_ bN}^{1} \\ {z}_{\_ bN}^{1}\end{matrix} ) \\ ( \begin{matrix}{x}_{\_ t}^{1} \\ {y}_{\_ t}^{1} \\ {z}_{\_ t}^{1}\end{matrix} ) = ( \begin{matrix}{x}_{2}^{′} \\ {y}_{2}^{′} \\ {z}_{2}^{′}\end{matrix} ) + ( \begin{matrix}{m}_{00}^{2} & {m}_{01}^{2} & {m}_{02}^{2} \\ {m}_{10}^{2} & {m}_{11}^{2} & {m}_{12}^{2} \\ {m}_{20}^{2} & {m}_{21}^{2} & {m}_{22}^{2}\end{matrix} ) ( \begin{matrix}Δ x \\ Δ y \\ Δ z\end{matrix} ) + ( \begin{matrix}{m}_{00}^{2} & {m}_{01}^{2} & {m}_{02}^{2} \\ {m}_{10}^{2} & {m}_{11}^{2} & {m}_{12}^{2} \\ {m}_{20}^{2} & {m}_{21}^{2} & {m}_{22}^{2}\end{matrix} ) ( \begin{matrix}{r}_{00} & {r}_{01} & {r}_{02} \\ {r}_{10} & {r}_{11} & {r}_{12} \\ {r}_{20} & {r}_{21} & {r}_{22}\end{matrix} ) ( \begin{matrix}{x}_{\_ b 1}^{2} \\ {y}_{\_ b 1}^{2} \\ {z}_{\_ b 1}^{2}\end{matrix} ) \\ ( \begin{matrix}{x}_{\_ t}^{2} \\ {y}_{\_ t}^{2} \\ {z}_{\_ t}^{2}\end{matrix} ) = ( \begin{matrix}{x}_{2}^{′} \\ {y}_{2}^{′} \\ {z}_{2}^{′}\end{matrix} ) + ( \begin{matrix}{m}_{00}^{2} & {m}_{01}^{2} & {m}_{02}^{2} \\ {m}_{10}^{2} & {m}_{11}^{2} & {m}_{12}^{2} \\ {m}_{20}^{2} & {m}_{21}^{2} & {m}_{22}^{2}\end{matrix} ) ( \begin{matrix}Δ x \\ Δ y \\ Δ z\end{matrix} ) + ( \begin{matrix}{m}_{00}^{2} & {m}_{01}^{2} & {m}_{02}^{2} \\ {m}_{10}^{2} & {m}_{11}^{2} & {m}_{12}^{2} \\ {m}_{20}^{2} & {m}_{21}^{2} & {m}_{22}^{2}\end{matrix} ) ( \begin{matrix}{r}_{00} & {r}_{01} & {r}_{02} \\ {r}_{10} & {r}_{11} & {r}_{12} \\ {r}_{20} & {r}_{21} & {r}_{22}\end{matrix} ) ( \begin{matrix}{x}_{\_ b 2}^{2} \\ {y}_{\_ b 2}^{2} \\ {z}_{\_ b 2}^{2}\end{matrix} ) \\ ………… \\ ( \begin{matrix}{x}_{\_ t}^{N} \\ {y}_{\_ t}^{N} \\ {z}_{\_ t}^{N}\end{matrix} ) = ( \begin{matrix}{x}_{2}^{′} \\ {y}_{2}^{′} \\ {z}_{2}^{′}\end{matrix} ) + ( \begin{matrix}{m}_{00}^{2} & {m}_{01}^{2} & {m}_{02}^{2} \\ {m}_{10}^{2} & {m}_{11}^{2} & {m}_{12}^{2} \\ {m}_{20}^{2} & {m}_{21}^{2} & {m}_{22}^{2}\end{matrix} ) ( \begin{matrix}Δ x \\ Δ y \\ Δ z\end{matrix} ) + ( \begin{matrix}{m}_{00}^{2} & {m}_{01}^{2} & {m}_{02}^{2} \\ {m}_{10}^{2} & {m}_{11}^{2} & {m}_{12}^{2} \\ {m}_{20}^{2} & {m}_{21}^{2} & {m}_{22}^{2}\end{matrix} ) ( \begin{matrix}{r}_{00} & {r}_{01} & {r}_{02} \\ {r}_{10} & {r}_{11} & {r}_{12} \\ {r}_{20} & {r}_{21} & {r}_{22}\end{matrix} ) ( \begin{matrix}{x}_{\_ bN}^{2} \\ {y}_{\_ bN}^{2} \\ {z}_{\_ bN}^{2}\end{matrix} ) \\ ………… \\ ( \begin{matrix}{x}_{\_ t}^{1} \\ {y}_{\_ t}^{1} \\ {z}_{\_ t}^{1}\end{matrix} ) = ( \begin{matrix}{x}_{p}^{′} \\ {y}_{p}^{′} \\ {z}_{p}^{′}\end{matrix} ) + ( \begin{matrix}{m}_{00}^{p} & {m}_{01}^{p} & {m}_{02}^{p} \\ {m}_{10}^{p} & {m}_{11}^{p} & {m}_{12}^{p} \\ {m}_{20}^{p} & {m}_{21}^{p} & {m}_{22}^{p}\end{matrix} ) ( \begin{matrix}Δ x \\ Δ y \\ Δ z\end{matrix} ) + ( \begin{matrix}{m}_{00}^{p} & {m}_{01}^{p} & {m}_{02}^{p} \\ {m}_{10}^{p} & {m}_{11}^{p} & {m}_{12}^{p} \\ {m}_{20}^{p} & {m}_{21}^{p} & {m}_{22}^{p}\end{matrix} ) ( \begin{matrix}{r}_{00} & {r}_{01} & {r}_{02} \\ {r}_{10} & {r}_{11} & {r}_{12} \\ {r}_{20} & {r}_{21} & {r}_{22}\end{matrix} ) ( \begin{matrix}{x}_{\_ b 1}^{p} \\ {y}_{\_ b 1}^{p} \\ {z}_{\_ b 1}^{p}\end{matrix} ) \\ ( \begin{matrix}{x}_{\_ t}^{2} \\ {y}_{\_ t}^{2} \\ {z}_{\_ t}^{2}\end{matrix} ) = ( \begin{matrix}{x}_{p}^{′} \\ {y}_{p}^{′} \\ {z}_{p}^{′}\end{matrix} ) + ( \begin{matrix}{m}_{00}^{p} & {m}_{01}^{p} & {m}_{02}^{p} \\ {m}_{10}^{p} & {m}_{11}^{p} & {m}_{12}^{p} \\ {m}_{20}^{p} & {m}_{21}^{p} & {m}_{22}^{p}\end{matrix} ) ( \begin{matrix}Δ x \\ Δ y \\ Δ z\end{matrix} ) + ( \begin{matrix}{m}_{00}^{p} & {m}_{01}^{p} & {m}_{02}^{p} \\ {m}_{10}^{p} & {m}_{11}^{p} & {m}_{12}^{p} \\ {m}_{20}^{p} & {m}_{21}^{p} & {m}_{22}^{p}\end{matrix} ) ( \begin{matrix}{r}_{00} & {r}_{01} & {r}_{02} \\ {r}_{10} & {r}_{11} & {r}_{12} \\ {r}_{20} & {r}_{21} & {r}_{22}\end{matrix} ) ( \begin{matrix}{x}_{\_ b 2}^{p} \\ {y}_{\_ b 2}^{p} \\ {z}_{\_ b 2}^{p}\end{matrix} ) \\ ………… \\ ( \begin{matrix}{x}_{\_ t}^{N} \\ {y}_{\_ t}^{N} \\ {z}_{\_ t}^{N}\end{matrix} ) = ( \begin{matrix}{x}_{p}^{′} \\ {y}_{p}^{′} \\ {z}_{p}^{′}\end{matrix} ) + ( \begin{matrix}{m}_{00}^{p} & {m}_{01}^{p} & {m}_{02}^{p} \\ {m}_{10}^{p} & {m}_{11}^{p} & {m}_{12}^{p} \\ {m}_{20}^{p} & {m}_{21}^{p} & {m}_{22}^{p}\end{matrix} ) ( \begin{matrix}Δ x \\ Δ y \\ Δ z\end{matrix} ) + ( \begin{matrix}{m}_{00}^{p} & {m}_{01}^{p} & {m}_{02}^{p} \\ {m}_{10}^{p} & {m}_{11}^{p} & {m}_{12}^{p} \\ {m}_{20}^{p} & {m}_{21}^{p} & {m}_{22}^{p}\end{matrix} ) ( \begin{matrix}{r}_{00} & {r}_{01} & {r}_{02} \\ {r}_{10} & {r}_{11} & {r}_{12} \\ {r}_{20} & {r}_{21} & {r}_{22}\end{matrix} ) ( \begin{matrix}{x}_{\_ bN}^{p} \\ {y}_{\_ bN}^{p} \\ {z}_{\_ bN}^{p}\end{matrix} ) \\ ………… \qquad ( 4 )$$
wherein:
p represents p^{th} time to get and record the center position data of N marker/markers at different p times position, and p=5 if N=1, p=3 if N=2 or N=3, and p=2 if N≥4; and
(x__{b}, y__{b}, z__{b}) represents a known center position data of the marker with respect to the coordinate system of the vision measuring system; and
(x′, y′, z′) represents a known position data of the tracking tool; and
a matrix
$$( \begin{matrix}{m}_{00} & {m}_{01} & {m}_{02} \\ {m}_{10} & {m}_{11} & {m}_{12} \\ {m}_{20} & {m}_{21} & {m}_{22}\end{matrix} )$$
is known for direction data of the tracking tool; and
(x^{p}__{bN}, y^{p}__{bN}, z^{p}__{bN}) represents a center position data of N^{th} marker on p^{th} position's recording; and
(xₚ′, yₚ′, zₚ′) represents a position data of the tracking tool on p^{th} position's recording; and
a matrix:
$$( \begin{matrix}{m}_{00}^{p} & {m}_{01}^{p} & {m}_{02}^{p} \\ {m}_{10}^{p} & {m}_{11}^{p} & {m}_{12}^{p} \\ {m}_{20}^{p} & {m}_{21}^{p} & {m}_{22}^{p}\end{matrix} )$$
is known for direction data of the tracking tool on p^{th} position's recording; and
(x^{N}_t, y^{N}_t, z^{N}_t) represents a center position of N^{th} marker with respect to the tracking assembly's coordinate system; and
(Δx, Δy, Δz) represents the position calibration offset between the coordinate system of the vision measuring system and the tracking tool; and
$$( \begin{matrix}{r}_{00} & {r}_{01} & {r}_{02} \\ {r}_{10} & {r}_{11} & {r}_{12} \\ {r}_{20} & {r}_{21} & {r}_{22}\end{matrix} )$$
represents the directional calibration relationship between the coordinate system of the vision measuring system and the tracking tool.

11. The method of claim 10, wherein the solving, based on the at least p groups of relative center position data of N marker/markers, nonhomogeneous linear equations to thereby obtain calibration relationship between the designated coordinate system of the vision measuring system and the tracking tool, wherein the nonhomogeneous linear equations are derived from the relationship between a center position of spherical marker with respect to the designated coordinate system of the vision measuring system and that position with respect to the coordinate system of the tracking assembly comprises:
solving the formula (4) of at least N×3×p equations in at least p×N equation groups to thereby obtain:
the position offset: (Δx, Δy, Δz); and
the matrix of direction calibration:
$$( \begin{matrix}{r}_{00} & {r}_{01} & {r}_{02} \\ {r}_{10} & {r}_{11} & {r}_{12} \\ {r}_{20} & {r}_{21} & {r}_{22}\end{matrix} ) .$$

12. The method of claim 1, wherein the marker for each target comprises a first portion and a second portion; and the first portion has a shape of a sphere and is substantially at a core center of the spherical marker; and the second portion is at an outer layer of the spherical marker and is arranged such that a core center of the second portion also substantially coincides with the core center of the first portion; and the first portion and the second portion have different compositions capable of generating a relatively either weak or strong signal compared each other by a diagnostic imaging scanner, as such, in the scanned imaging, the image position of center of the first portion of the marker can be determined and measured easily and accurately with distinguishingly displayed spot.

13. The method of claim 12, wherein the at least one target is the at least four targets and the method further comprising:
a) reconstituting, based on the at least four markers of targets, a group position data for an area comprising the at least four targets with respect to a reference coordinate system of the tracking assembly, wherein:
the at least four target positions are not coplanar in three-dimensional space; and
each target has a rigid fixed position relatively to each other; and
the origin and direction of the reference coordinate system of the tracking assembly is arranged at a rigid fixed position and direction relatively to the group positions of the at least four markers of targets;
b) scanning an object for navigation and the group of the at least four markers of targets together via an imaging scanner, to obtain a group of imaging position data of the at least four markers of targets, wherein the relative position and direction among the object for navigation, the origin and direction of the reference coordinate system of the tracking assembly and each of the at least four markers of targets are rigid fixed each other;
c) calculating, based on the two groups of position data in imaging world and that in physical world, the transformation of positions and directions between the physical word and the imaging world, which is used for navigation regarding the object, under the condition that the relative position and direction between the object and the origin and direction of the reference coordinate system of the tracking assembly are rigid fixed and unchanged from the above scanning step b).

14. The method of claim 13, wherein:
the tracking assembly comprises a transmitter configured to generate an electromagnetic field; and
the tracking tool comprises a sensing coil configured to produce an induced voltage in the electromagnetic field; and
the tracking assembly further comprises an electronics unit, is coupled to the sensing coil and the transmitter and is configured to calculate the position and orientation data of the tracking tool based on the induced voltage produced in the sensing coil; and
the reference coordinate system of the tracking assembly is bases on a tracking tool of six-degree of position and direction.
